(19) United States
(12) Patent Application Publication
(10) Pub. No.: US 2011/0110945 A1
(43) Pub. Date: May 12, 2011

(54) Immunoglobulin Fusion Proteins and Methods of Making

(75) Inventors: Adnan M.M. Mjalli, Oak Ridge, NC (US); Ye Edward Tian, Jamestown, NC (US); Jeffrey C. Webster, Jamestown, NC (US); Robert Rothlein, Summerfield, NC (US)

(73) Assignee: TransTech Pharma, Inc., High Point, NC (US)

(21) Appl. No.: 12/899,073

(22) Filed: Oct. 6, 2010

Related U.S. Application Data
(62) Division of application No. 12/069,499, filed on Feb. 11, 2008, now abandoned.
(60) Provisional application No. 60/901,540, filed on Feb. 15, 2007.

(51) Int. Cl. A61K 39/395; C07K 16/00; A61P 43/00

(52) U.S. Cl. 424/134.1; 530/387.3

(57) ABSTRACT

Disclosed are immunoglobulin fusion proteins and methods of making such proteins. In certain embodiments, the fusion protein may include a non-immunoglobulin polypeptide linked to an immunoglobulin polypeptide. In certain embodiments, the non-immunoglobulin polypeptide may comprise a region that replaces an immunoglobulin Fc hinge region, but that allows for correct assembly of the immunoglobulin chains.

### CROSS REFERENCE TO RELATED APPLICATIONS

The present application is a divisional of U.S. application Ser. No. 12/069,499, filed Feb. 11, 2008, which claims priority under 35 USC 119(e) to U.S. Provisional Patent Application Ser. No. 60/901,540 filed Feb. 15, 2007. The disclosure of U.S. Provisional Patent Application 60/901,540 is hereby incorporated by reference in its entirety herein.

### FIELD OF THE INVENTION

The present invention relates to immunoglobulin fusion proteins and methods of making such proteins.

### BACKGROUND

Fusion proteins may be used as a means to provide a biomolecule that includes a first polypeptide of interest covalently linked to a second polypeptide. The second polypeptide may provide a means for delivery of the first polypeptide to a site of interest (e.g., a membrane-bound receptor). Alternatively, the second polypeptide may provide a means for activating the first polypeptide. For example, U.S. Pat. No. 5,843,725 describes linking receptor molecules that require dimerization for biological activity to immunoglobulin heavy and light chains as a means to generate receptor dimers.

The second polypeptide may provide a means for stabilizing the first polypeptide in an organism. For example, peptides may be linked to immunoglobulin domains as a way to enhance the bioactivity and/or physiological half-life of the peptides. However, certain portions of the immunoglobulin, such as the portion of the Fc hinge domain that can bind to the Fc receptor (FcR), may initiate an inflammatory response and thus, can lead to an adverse reaction when the fusion protein is administered over the long-term. Thus, it would be useful to develop fusion proteins that do not include portions of the immunoglobulin that elicit an inflammatory response as a means to decrease clearance of the fusion protein via the Fc receptor or other mechanisms.

### SUMMARY

Embodiments of the present invention comprise immunoglobulin fusion proteins and methods of making such proteins. The present invention may be embodied in a variety of ways.

In some embodiments, the fusion protein may comprise a non-immunoglobulin polypeptide linked to an immunoglobulin polypeptide. In certain embodiments, the non-immunoglobulin polypeptide may comprise a region, as for example, an interdomain linker, which replaces the immunoglobulin Fc hinge region in a manner that allows for correct assembly of the immunoglobulin polypeptide. For example, in an embodiment, the present invention may comprise a fusion protein comprising: (a) an immunoglobulin polypeptide from which the hinge region has been removed, and (b) a non-immunoglobulin polypeptide.

The non-immunoglobulin polypeptide may, in certain embodiments, comprise an uninterrupted portion of an immunoglobulin supergene protein. In some embodiments, the uninterrupted portion of the immunoglobulin supergene protein may comprise a first non-immunoglobulin polypeptide domain directly linked to the N-terminal end of a first interdomain linker. Also in some embodiments, the immunoglobulin polypeptide may be operably linked to the C-terminal end of the first interdomain linker.

In some embodiments, the interdomain linker sequence from the non-immunoglobulin polypeptide does not promote binding of the fusion protein to a Fc receptor (FcR) at therapeutically effective concentrations of the fusion protein. In certain embodiments, the immunoglobulin polypeptide may comprise an Fc fragment without the hinge region. In one embodiment, the immunoglobulin polypeptide comprises at least one constant domain or a portion of a constant domain. In certain embodiments, the immunoglobulin polypeptide may comprise at least one of the C_{H}2 domain or the C_{H}3 domain of an immunoglobulin, or at least one of a portion of the C_{H}2 domain or a portion of the C_{H}3 domain of an immunoglobulin.

Embodiments of the present invention also comprise methods to make a fusion protein. In one embodiment, the method comprises covalently linking a non-immunoglobulin polypeptide to an immunoglobulin polypeptide. In certain embodiments, the non-immunoglobulin polypeptide may comprise a region, as for example, an interdomain linker, which replaces the immunoglobulin hinge region in a manner that allows for correct assembly of the immunoglobulin polypeptide. For example, in certain embodiments, the present invention may comprise methods to make a fusion protein comprising: linking (a) an immunoglobulin polypeptide from which the hinge region has been removed, and (b) a non-immunoglobulin polypeptide.

The non-immunoglobulin polypeptide may, in certain embodiments, comprise an uninterrupted portion of an immunoglobulin supergene protein. In some embodiments, the uninterrupted portion of the immunoglobulin supergene protein may comprise a first non-immunoglobulin polypeptide domain directly linked to the N-terminal end of a first interdomain linker. Also in some embodiments, the immunoglobulin polypeptide may be operably linked to the C-terminal end of the first interdomain linker. In certain embodiments, the immunoglobulin polypeptide may comprise an Fc fragment without the hinge region. In an embodiment, the immunoglobulin polypeptide comprises at least one constant domain or a portion thereof. In certain embodiments, the immunoglobulin polypeptide may comprise at least one of an immunoglobulin C_{H}2 domain or a portion thereof and/or an immunoglobulin C_{H}3 domain or a portion thereof.

Embodiments of the present invention may also comprise a composition comprising a formulation of a fusion protein of the present invention that is suitable for administration to a subject. In an embodiment, the formulation may be lyophilized.

There are various advantages that may be associated with particular embodiments of the present invention. As noted above, in some embodiments, the interdomain linker sequence from the non-immunoglobulin polypeptide may not promote binding of the fusion protein to a Fc receptor (FcR) at therapeutically effective concentrations of the fusion protein. Thus, in some embodiments, the fusion proteins of the present invention may induce less inflammation when administered to a subject as compared to fusion proteins having functional Fc hinge sequences.

Also, where the non-immunoglobulin polypeptide comprises a ligand binding domain or ligand binding site such as a receptor, the fusion proteins of the present invention may exhibit high-affinity binding to ligands, thereby providing a pharmacological agent that may be used to treat diseases that are mediated by the receptor and/or ligand(s). In certain embodiments, the fusion proteins of the present invention bind to ligands with affinities in the high nanomolar to low micromolar range. By binding with high affinity to physiological ligands, the fusion proteins of the present invention may be used to inhibit binding of endogenous ligands to receptors, thereby providing a means to ameliorate diseases.

Also, the fusion proteins of the present invention may be provided in protein or nucleic acid form. In one example embodiment, the fusion protein may be administered systemically and remain in the vasculature to potentially treat vascular diseases. In another example embodiment, the fusion protein may be administered locally to treat diseases where the non-immunoglobulin polypeptide, or compounds that interact with the non-immunoglobulin polypeptide, are involved at least in part in the pathology of the disease. Alternatively, a nucleic acid construct encoding the fusion protein may be delivered to a site (e.g., a cell or tissue type of interest) by the use of an appropriate carrier such as a virus, or as a naked DNA, where transient or long-term local expression of the non-immunoglobulin polypeptide may locally inhibit the interaction between the polypeptide encoded by the fusion protein and a physiological biomolecule. Thus, administration may be transient (e.g., as where the fusion protein is administered) or more permanent in nature (e.g., as where the fusion protein is administered as a recombinant DNA).

There are additional features of the invention which will be described hereinafter. It is to be understood that the invention is not limited in its application to the details set forth in the following claims, description and figures. The invention is capable of other embodiments and of being practiced or carried out in various ways.

### BRIEF DESCRIPTION OF THE FIGURES

Various features, aspects and advantages of the present invention will become more apparent with reference to the following figures.

FIG. 1 shows various sequences and immunoglobulin sequences in accordance with alternate embodiments of the present invention: Panel A, SEQ ID NO: 1, an amino acid sequence for human RAGE; SEQ ID NO: 2, a DNA encoding human RAGE amino acids 1-251; Panel B, SEQ ID NO: 3, a RAGE interdomain linker; SEQ ID NO: 4, a second RAGE interdomain linker; SEQ ID NO: 5, a third RAGE interdomain linker; SEQ ID NO: 6, a fourth RAGE interdomain linker; SEQ ID NO: 7, a fifth RAGE interdomain linker; Panel C, SEQ ID NO: 8, an amino acid sequence for the C_{H}2 and C_{H}3 domains of human IgG without the hinge region; SEQ ID NO: 9 and SEQ ID NO: 10, alternate DNA sequences encoding a portion of the human C_{H}2 and C_{H}3 domains of human IgG without the hinge region where SEQ ID NO: 10 is modified from SEQ ID NO: 9 to remove a cryptic RNA splice site and the modification does not change the amino acid sequence of the protein; Panel D, SEQ ID NO: 11, an amino acid sequence for the C_{H}2 and C_{H}3 domains of human IgG, which includes a portion of the middle hinge and the lower hinge region; SEQ ID NO: 12 and SEQ ID NO: 13, alternate DNA sequences encoding the human C_{H}2 and C_{H}3 domains of human IgG, which include a portion of the middle hinge and the lower hinge region, where SEQ ID NO: 13 is modified from SEQ ID NO: 12 to remove a cryptic RNA splice site and the modification does not change the amino acid sequence of the protein; Panel E, SEQ ID NO: 14, an amino acid sequence for the C_{H}2 domain of human IgG without the lower (and middle) hinge region; and SEQ ID NO: 15, an amino acid sequence for the C_{H}3 domain of human IgG.

FIG. 2 shows a schematic representation of alternate fusion proteins comprising a non-immunoglobulin polypeptide that has a region, as for example, an interdomain linker, which replaces the immunoglobulin hinge region in a manner that allows for correct assembly of a immunoglobulin polypeptide, and where the non-immunoglobulin polypeptide is linked to at least one immunoglobulin polypeptide in accordance with alternate embodiments (2a-2e) of the present invention.

FIG. 3, Panel A, shows a comparison of the protein domains in human RAGE and human Ig gamma-1 Fc protein, and cleavage points used to make TTP-3000 at position 136 of human RAGE (hRAGE), and TTP-4000 at position 251 of hRAGE, in accordance with alternate embodiments of the present invention; and Panel B shows the three domain structure for TTP-3000 and the four domain structure for TTP-4000 in accordance with alternate embodiments of the present invention.

FIG. 4 shows amino acid sequences SEQ ID NO: 16 (Panel A) for a four domain fusion protein, SEQ ID NO: 17 (Panel B) for a three domain fusion protein, SEQ ID NO: 18 (Panel C) for a four domain fusion protein having a signal sequence removed and the N-terminal glutamine cyclized to a pyroglutamic acid, and SEQ ID NO: 19 (Panel D) for a three domain fusion protein having a signal sequence removed and the N-terminal glutamine cyclized to a pyroglutamic acid in accordance with alternate embodiments of the present invention. The non-immunoglobulin sequence is highlighted with bold font.

FIG. 5 shows two alternate the DNA sequences (SEQ ID NO: 20) (Panel A) and SEQ ID NO: 21 (Panel B) that each encode a four domain fusion protein in accordance with an embodiment of the present invention. Coding sequence 1-753 highlighted in bold encodes a non-immunoglobulin N-terminal protein sequence whereas sequence 754-1386 encodes human IgG (γ1) protein sequence. SEQ ID NO: 21 is modified from SEQ ID NO: 20 to remove a cryptic RNA splice site; the modification does not change the amino acid sequence of the protein.

FIG. 6 shows two alternate the DNA sequences (SEQ ID NO: 22) (Panel A) and SEQ ID NO: 23 (Panel B) that each encode a three domain fusion protein in accordance with an embodiment of the present invention. Coding sequence 1-408 highlighted in bold encodes a non-immunoglobulin N-terminal protein sequence, whereas sequence 409-1041 codes human IgG (γ1) protein sequence. SEQ ID NO: 23 is modified from SEQ ID NO: 22 to remove a cryptic RNA splice site; the modification does not change the amino acid sequence of the protein.

FIG. 7 shows results of an in vitro binding assay for sRAGE, and a RAGE fusion proteins TTP-3000 (TT3) and TTP-4000 (TT4) to the RAGE ligands amyloid-beta (A-beta), S100b (S100), and amphoterin (Ampho), in accordance with alternate embodiments of the present invention.

FIG. 8 shows results of an in vitro binding assay for a RAGE fusion protein TTP-4000 (TT4) (“Protein”) to amyloid-beta as compared to a negative control only including the immunodetection reagents (“Complex Alone”), and antagonism of such binding by a RAGE antagonist (“RAGE Ligand”) in accordance with alternate embodiments of the present invention.

FIG. 9 shows results of an in vitro binding assay for RAGE fusion protein TTP-3000 (TT3) (“Protein”) to amyloid-beta as compared to a negative control only including the immunodetection reagents (“Complex Alone”), and antagonism of such binding by a RAGE antagonist (“RAGE Ligand”) in accordance with alternate embodiments of the present invention.

FIG. 10 shows results of a cell-based assay measuring the inhibition of S100b-RAGE induced production of TNF-α by RAGE fusion proteins TTP-3000 (TT3) and TTP-4000 (TT4) and sRAGE in accordance with alternate embodiments of the present invention.

FIG. 11 shows a pharmacokinetic profile for RAGE fusion protein TTP-4000 in accordance with one embodiment of the present invention wherein each curve represents a different animal under the same experimental conditions.

FIG. 12 shows relative levels of TNF-α release from THP-1 cells due to stimulation by RAGE fusion protein TTP-4000 and human IgG stimulation as a measure of an inflammatory response in accordance with one embodiment of the present invention.

FIG. 13 shows saturation-binding curves with TTP-4000 to various immobilized known RAGE ligands in accordance with alternate embodiments of the present invention.

### DETAILED DESCRIPTION

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, the terms “portion” and “fragment” are used interchangeably to refer to parts of a polypeptide, nucleic acid, or other molecular construct.

“Polypeptide” and “protein” are used interchangeably herein to describe protein molecules that may comprise either partial or full-length proteins. The term “peptide” is used to denote a less than full-length protein or a very short protein unless the context indicates otherwise.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

As used herein, the term “upstream” refers to a residue that is N-terminal to a second residue where the molecule is a protein, or 5′ to a second residue where the molecule is a nucleic acid. Also as used herein, the term “downstream” refers to a residue that is C-terminal to a second residue where the molecule is a protein, or 3′ to a second residue where the molecule is a nucleic acid. The protein, polypeptide and peptide sequences disclosed herein are all listed from N-terminal amino acid to C-terminal acid and the nucleic acid sequences disclosed herein are all listed from the 5′ end of the molecule to the 3′ end of the molecule.

Unless defined otherwise, all technical and scientific terms used herein have the same meaning as commonly understood by one of ordinary skill in the art. Practitioners are particularly directed to Current Protocols in Molecular Biology (Ausubel) for definitions and terms of the art. Abbreviations for amino acid residues are the standard 3-letter and/or 1-letter codes used in the art to refer to one of the 20 common L-amino acids.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

The term “vector” refers to a nucleic acid molecule that may be used to transport a second nucleic acid molecule into a cell. In one embodiment, the vector allows for replication of DNA sequences inserted into the vector. The vector may comprise a promoter to enhance expression of the nucleic acid molecule in at least some host cells. Vectors may replicate autonomously (extrachromasomal) or may be integrated into a host cell chromosome. In one embodiment, the vector may comprise an expression vector capable of producing a protein derived from at least part of a nucleic acid sequence inserted into the vector.

As is known in the art, conditions for hybridizing nucleic acid sequences to each other can be described as ranging from low to high stringency. Generally, highly stringent hybridization conditions refer to washing hybrids in low salt buffer at high temperatures. Hybridization may be to filter bound DNA using hybridization solutions standard in the art such as 0.5M NaHPO₄, 7% sodium dodecyl sulfate (SDS), at 65° C., and washing in 0.25 M NaHPO₄, 3.5% SDS followed by washing 0.1×SSC/0.1% SDS at a temperature ranging from room temperature to 68° C. depending on the length of the probe (see e.g. Ausubel, F. M. et al., Short Protocols in Molecular Biology, 4^{th} Ed., Chapter 2, John Wiley & Sons, N.Y). For example, a high stringency wash comprises washing in 6×SSC/0.05% sodium pyrophosphate at 37° C. for a 14 base oligonucleotide probe, or at 48° C. for a 17 base oligonucleotide probe, or at 55° C. for a 20 base oligonucleotide probe, or at 60° C. for a 25 base oligonucleotide probe, or at 65° C. for a nucleotide probe about 250 nucleotides in length. Nucleic acid probes may be labeled with radionucleotides by end-labeling with, for example, [γ-³²P]ATP, or incorporation of radiolabeled nucleotides such as [α-³²P]dCTP by random primer labeling. Alternatively, probes may be labeled by incorporation of biotinylated or fluorescein labeled nucleotides, and the probe detected using Streptavidin or anti-fluorescein antibodies.

The term “fusion protein” refers to a protein or polypeptide that has an amino acid sequence derived from two or more proteins. The fusion protein may also include linking regions of amino acids between amino acid portions derived from separate proteins.

As used herein, “RAGE” or “Receptor for Advanced Glycation Endproducts” is a multiligand receptor that is a member of the immunoglobulin (Ig) superfamily. As used herein, RAGE includes wild-type human RAGE (M91211), as well as functional RAGE variants and/or isoforms (see e.g., WO 2005/051995) The amino acid sequences of the RAGE isoforms described in WO 2005/051995 and other known RAGE isoforms are incorporated by reference in their entireties herein. RAGE proteins may contain a single membrane-spanning domain of about 19 amino acids, which separates the protein into an extracellular domain and an intracellular domain (Neeper, M. et al., J. Biol. Chem. 267(21):14998-15004 (1992)). In the case of human wild-type RAGE, the protein is 404 amino acids in length and comprises a signal sequence of about 19-23 amino acids, an extracellular domain of about 321 amino acids, a transmembrane domain of about 23 amino acids and an intracellular domain of about 41 amino acids (Neeper, M. et al., 1992). The nucleotide sequence is available in public databases such as GenBank Accession No. M91211, and the amino acid sequence is available as GenBank Accession No. NP 001127. RAGE proteins from other species contain similar domains, although not necessarily of the same length. RAGE contains three immunoglobulin-like domains (Ig-like domains) (Neeper et al., 1992). The N-terminal Ig-like domain is a V-like domain and, in human RAGE, this V-like domain spans from about amino acid 24 to 118.

As used herein, a “non-immunoglobulin polypeptide” is any polypeptide that is not derived from an immunoglobulin or a fragment thereof. In certain embodiments, the non-immunoglobulin polypeptide is an uninterrupted polypeptide derived from a polypeptide that is not derived from an immunoglobulin or a fragment thereof. In one embodiment, a “non-immunoglobulin polypeptide” is a polypeptide that is not derived from an immunoglobulin or a fragment thereof but is a member of the immunoglobulin supergene family. Such non-immunoglobulin polypeptides that are not derived from an immunoglobulin but are a member of the immunoglobulin supergene family include polypeptides that have immunoglobulin-like domains, such as, but not limited to, the members of the immunoglobulin supergene family described herein. Such peptides may, in certain embodiments, comprise a ligand binding domain or ligand binding site.

As used herein, a “non-immunoglobulin polypeptide domain” is any polypeptide domain that is not derived from an immunoglobulin or a fragment thereof. In certain embodiments, the non-immunoglobulin polypeptide domain is an uninterrupted polypeptide derived from any polypeptide that is not derived from an immunoglobulin or a fragment thereof. In one embodiment, a “non-immunoglobulin polypeptide domain” is a polypeptide domain that is not derived from an immunoglobulin or a fragment thereof but is derived from a member of the immunoglobulin supergene family. Such non-immunoglobulin polypeptide domains that are not derived from an immunoglobulin but are derived from a member of the immunoglobulin supergene family include polypeptide domains from members of the immunoglobulin supergene family described herein. Such non-immunoglobulin polypeptide domains may comprise a ligand binding domain or ligand binding site or a portion of a ligand binding domain or ligand binding site. In certain embodiments, the immunoglobulin polypeptide domains may comprise an uninterrupted portion of a ligand binding domain or an uninterrupted portion of ligand binding site.

As used herein, the terms “immunoglobulin superfamily” or “immunoglobulin supergene family” are used interchangeably herein to refer to a large group of cell surface and soluble proteins that are involved in the immune system. Such proteins may be involved in recognition, binding, or cell adhesion processes. Proteins are included as members of this superfamily based on structural features that are shared with immunoglobulins. Members of the immunoglobulin superfamily include cell surface antigen receptors, co-receptors and co-stimulatory molecules, and molecules involved in antigen presentation. Other types of proteins that are considered to be members of the immunoglobulin superfamily include cell adhesion molecules and certain cytokine receptors. Proteins are classified into the immunoglobulin superfamily if they possess an immunoglobulin-like domain. In addition to immunoglobulins (or antibodies), members of the immunoglobulin superfamily include RAGE, T cell receptors, Killer-cell immunoglobulin-like receptors (KIR), proteins that comprise the class I or class II major histocompatibility complex (MHC class I), CD2, the CD3 receptor (γ, δ and ε chains), CD4, CD8, CD28, CD79a and CD79b, CD80 and CD86 (i.e., B7.1 and B7.2), CD147, LFA, and CTLA4, certain Fc receptors, Intercellular adhesion molecules (ICAMs), Vascular cell adhesion molecule-1 (VCAM-1), Neural Cell Adhesion Molecule (NCAM), Interleukin-1 receptor type I, Platelet-derived growth factor receptor (PDGFR), and Thymocyte differentiation antigen-1 (i.e., Thy-1 or CD90).

As used herein, “immunoglobulin polypeptide(s)” is a polypeptide derived from an immunoglobulin. In some embodiments, the immunoglobulin polypeptide may comprise an uninterrupted native sequence of an immunoglobulin. In some embodiments, an immunoglobulin polypeptide may comprise an immunoglobulin heavy chain or a portion thereof. In one embodiment, the portion of the heavy chain may be the Fc fragment or a portion thereof. As used herein, the Fc fragment comprises the heavy chain hinge polypeptide, and the C_{H}2 and C_{H}3 domains of the heavy chain of an immunoglobulin, in either monomeric or dimeric form. Or, the C_{H}1 and Fc fragment may be used as the immunoglobulin polypeptide. The heavy chain (or portion thereof) may be derived from any one of the known heavy chain isotypes: IgG (γ), IgM (μ), IgD (δ), IgE (ε), or IgA (α). In addition, the heavy chain (or portion thereof) may be derived from any one of the known heavy chain isotypes or subtypes: IgG1 (γ1), IgG2 (γ2), IgG3 (γ3), IgG4 (γ4), IgA1 (α1), IgA2 (α2), or mutations of these isotypes or subtypes that alter the biological activity.

As used herein, the hinge region is a region of an immunoglobulin that is positioned between, and may at least partially overlap, the heavy chain constant domains (e.g., the C_{H}2 and C_{H}1 exons), but that provides a flexible secondary structure between the immunoglobulin constant domains. For IgG, a hinge region is positioned between the C_{H}1 and C_{H}2 domains.

As used herein, the term “from which the hinge region has been removed” means that the amino acid sequences encoding the hinge region are excised from an immunoglobulin polypeptide sequence and replaced with a polypeptide that is derived from a non-immunoglobulin polypeptide. In an embodiment, the polypeptide derived from a non-immunoglobulin polypeptide that replaces the hinge region is an uninterrupted sequence of a native or naturally occurring non-immunoglobulin polypeptide.

The Ig fusion proteins of the invention include fusion proteins wherein the first non-immunoglobulin protein or fragment thereof is N-terminal to the immunoglobulin element, as well as fusion proteins wherein the immunoglobulin element is N-terminal to the non-immunoglobulin polypeptide or fragment thereof. In yet other embodiments, the fusion protein may comprise non-immunoglobulin domains that are positioned both N-terminal and C-terminal to an immunoglobulin domain or immunoglobulin domains that are positioned both N-terminal and C-terminal to a non-immunoglobulin domain.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In other cases, commercially available software, such as GenomeQuest, may be used to determine percent identity. When utilizing BLAST and Gapped BLAST programs, the default parameters of the respective programs (e.g., BLASTN; available at the Internet site for the National Center for Biotechnology Information) may be used. In one embodiment, the percent identity of two sequences may be determined using GCG with a gap weight of 1, such that each amino acid gap is weighted as if it were a single amino acid mismatch between the two sequences. Or, the ALIGN program (version 2.0), which is part of the GCG (Accelrys, San Diego, Calif.) sequence alignment software package may be used.

The binding properties of a fusion protein where the non-immunoglobulin portion of the protein comprises either a receptor or a ligand can be expressed in terms of binding specificity, which may be determined as a comparative measure relative to other known substances that bind to the receptor. Standard assays for quantifying binding and determining binding affinity are known in the art and include, e.g., equilibrium dialysis, equilibrium binding, gel filtration, surface plasmon resonance, the use of a labeled binding partners, ELISAs and indirect binding assays (e.g., competitive inhibition assays). For example, as is well known in the art, the dissociation constant of a protein can be determined by contacting the protein with a binding partner and measuring the concentration of bound and free protein as function of its concentration.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, the term “similar” or “homologue” when referring to amino acid or nucleotide sequences means a polypeptide having a degree of homology or identity with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 70% identical, 75% identical, 80% identical, 85% identical, 90% identical, 95% identical, 97% identical, or 98% identical to each other.

As used herein, the term at least 90% identical thereto includes sequences that range from 90 to 100% identity to the indicated sequences and includes all ranges in between. Thus, the term at least 90% identical thereto includes sequences that are 91, 91.5, 92, 92.5, 93, 93.5. 94, 94.5, 95, 95.5, 96, 96.5, 97, 97.5, 98, 98.5, 99, 99.5 percent identical to the indicated sequence. Similarly the term “at least 70% identical includes sequences that range from 70 to 100% identical, with all ranges in between. The determination of percent identity is determined using the algorithms described herein.

As used herein, a polypeptide or protein “domain” comprises a region along a polypeptide or protein that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS.

As used herein, an interdomain linker is a polypeptide sequence that connects one polypeptide domain to another polypeptide domain. The linker does not function as part of either domain, but connects the two domains in a neutral manner such that the two connected domains may have the proper structure or function as required.

As used herein, “directly linked” identifies a covalent linkage between two different groups (e.g., nucleic acid sequences, polypeptides, polypeptide domains) that does not have any intervening atoms between the two groups that are being linked.

As used herein, “immunoglobulin domain” is a sequence of amino acids that is structurally homologous, or identical to, a domain of an immunoglobulin. The length of the sequence of amino acids of an immunoglobulin domain may be any length. In one embodiment, an immunoglobulin domain may be less than 250 amino acids. In an example embodiment, an immunoglobulin domain may be about 80-150 amino acids in length. For example, the variable region, and the C_{H}1, C_{H}2, and C_{H}3 regions of an IgG are each immunoglobulin domains. In another example, the variable, the C_{H}1, C_{H}2, C_{H}3 and C_{H}4 regions of an IgM are each immunoglobulin domains.

Immunoglobulin domains may generally contain about 70-110 amino acids and are generally classified according to their size and function. They possess a characteristic fold in which two beta sheet of antiparallel beta strands form a sandwich type structure. The Ig-fold may be stabilized by interactions between charged amino acids on the inward face of the beta sheet, with further stabilization provided by disulphide bonds formed between cysteine residues. The major types of immunoglobulin domains are the variable domain and the constant domains.

Also as used herein, an immunoglobulin domain (or polypeptide domain) is not limited to an immunoglobulin from any particular species. Immunoglobulin domains may be derived from immunoglobulins from a variety of species. Thus, the immunoglobulin domain may be a mammalian immunoglobulin domain (i.e., an immunoglobulin domain derived from a mammalian immunoglobulin), such as a human immunoglobulin domain (i.e., an immunoglobulin domain derived from a human immunoglobulin). Immunoglobulin domains that are derived from immunoglobulins from other species, including, but not limited to, primates, cows, sheep, goats, horses, dogs, cats, rabbits, guinea pigs, rats, mice or other bovine, ovine, equine, canine, feline, rodent, or murine species, are also encompassed by the invention.

As used herein, a “RAGE domain” is a sequence of amino acids from RAGE protein that comprises an independent unit in terms of structure, sequence or function. For example, a RAGE domain may comprise the RAGE V-domain, the RAGE Ig-like C1-type 1 domain (“C1 domain”), or the RAGE Ig-like C2-type 2 domain (“C2 domain”), or functional variants or isoforms of such domains.

As used herein, “ligand binding domain” refers to a domain of a protein responsible for binding a ligand. The term ligand binding domain includes homologues of a ligand binding domain or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding domain is retained.

As used herein, a “ligand binding site” comprises residues in a protein that directly interact with a ligand, or residues involved in positioning the ligand in close proximity to those residues that directly interact with the ligand. The interaction of residues in the ligand binding site may be defined by the spatial proximity of the residues to a ligand in the model or structure. The term ligand binding site includes homologues of a ligand binding site, or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding site is retained. A ligand binding site may exist in one or more ligand binding domains of a protein or polypeptide.

As used herein, the term “interact” refers to a condition of proximity between a ligand or compound, or portions or fragments thereof, and a portion of a second molecule of interest. The interaction may be non-covalent, for example, as a result of hydrogen-bonding, van der Waals interactions, or electrostatic or hydrophobic interactions, or it may be covalent.

As used herein, a “ligand” refers to a molecule or compound or entity that interacts with a ligand binding site, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect. Ligands may include, but are not limited to, small molecule inhibitors. These small molecules may include peptides, peptidomimetics, organic compounds and the like. Ligands may also include polypeptides and/or proteins.

As used herein, a “modulator compound” refers to a molecule which changes or alters the biological activity of a molecule of interest. A modulator compound may increase or decrease activity, or change the physical or chemical characteristics, or functional or immunological properties, of the molecule of interest. A modulator compound may increase or decrease activity, or change the characteristics, or functional or immunological properties of a biomolecule of interest. A modulator compound may include natural and/or chemically synthesized or artificial peptides, modified peptides (e.g., phosphopeptides), antibodies, carbohydrates, monosaccharides, oligosaccharides, polysaccharides, glycolipids, heterocyclic compounds, nucleosides or nucleotides or parts thereof, and small organic or inorganic molecules. A modulator compound may be an endogenous physiological compound or it may be a natural or synthetic compound. Or, the modulator compound may be a small organic molecule. The term “modulator compound” also includes a chemically modified ligand or compound, and includes isomers and racemic forms.

An “agonist” comprises a compound that binds to a receptor to form a complex that elicits a pharmacological response specific to the receptor involved.

An “antagonist” comprises a compound that binds to an agonist or to a receptor to form a complex that does not give rise to a substantial pharmacological response and can inhibit the biological response induced by an agonist.

The term “peptide mimetics” refers to structures that serve as substitutes for peptides in interactions between molecules (Morgan et al., 1989, Ann. Reports Med. Chem., 24:243-252). Peptide mimetics may include synthetic structures that may or may not contain amino acids and/or peptide bonds but that retain the structural and functional features of a peptide, or agonist, or antagonist. Peptide mimetics also include peptoids, oligopeptoids (Simon et al., 1972, Proc. Natl. Acad, Sci., USA, 89:9367); and peptide libraries containing peptides of a designed length representing all possible sequences of amino acids corresponding to a peptide, or agonist or antagonist of the invention.

The term “treating”, “treatment”, or “treat” refers to improving a symptom of a disease or disorder, substantially delaying the onset of the disorder, or improving the subject's condition.

As used herein, the term “EC50” is defined as the concentration of an agent that results in 50% of a measured biological effect. For example, the EC50 of a therapeutic agent having a measurable biological effect may comprise the value at which the agent displays 50% of the biological effect.

As used herein, the term “IC50” is defined as the concentration of an agent that results in 50% inhibition of a measured effect. For example, the IC50 of an antagonist of binding may comprise the value at which the antagonist reduces ligand binding to a ligand binding site by 50%.

As used herein, an “effective amount” means the amount of an agent that is effective for producing a desired effect in a subject. The term “therapeutically effective amount” denotes that amount of a drug or pharmaceutical agent that will elicit therapeutic response of an animal or human that is being sought. The actual dose which comprises the effective amount may depend upon the route of administration, the size and health of the subject, the disorder being treated, and the like.

The term “pharmaceutically acceptable carrier” as used herein may refer to compounds and compositions that are suitable for use in human or animal subjects, as for example, for therapeutic compositions administered for the treatment of a disorder or disease of interest.

The term “pharmaceutical composition” is used herein to denote a composition that may be administered to a mammalian host, e.g., orally, parenterally, topically, by inhalation spray, intranasally, or rectally, in unit dosage formulations containing conventional non-toxic carriers, diluents, adjuvants, vehicles and the like.

The term “parenteral” as used herein, includes subcutaneous injections, intravenous, intramuscular, intracisternal injection, or infusion techniques.

As used herein “rejection” refers to the immune or inflammatory response on tissue that leads to destruction of cells, tissues or organs, or that leads to damage to cells, tissues, or organs. The rejected cells, tissue, or organ may be derived from the same subject that is mounting the rejection response, or may be transplanted from a different subject into the subject that is displaying rejection.

As used herein, the term “cell” refers to the structural and functional units of a mammalian living system that each comprise an independent living system. As is known in the art, cells include a nucleus, cytoplasm, intracellular organelles, and a cell membrane which encloses the cell and allows the cell to be independent of other cells.

As used herein, the term “tissue” refers to an aggregate of cells that have a similar structure and function, or that work together to perform a particular function. A tissue may include a collection of similar cells and the intercellular substances surrounding the cells. Tissues include, but are not limited to, muscle tissue, nerve tissue, and bone.

As used herein an “organ” refers to a fully differentiated structural and functional unit in an animal that is specialized for some specific function. An organ may comprise a group of tissues that perform a specific function or group of functions. Organs include, but are not limited to, the heart, lungs, brain, eye, stomach, spleen, pancreas, kidneys, liver, intestines, skin, uterus, bladder, and bone.

A “stable” formulation is one in which the fusion protein therein essentially retains its physical and chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 1 week to 1 month, at which time stability is measured. The extent of aggregation following lyophilization and storage can be used as an indicator of fusion protein stability. For example, a “stable” formulation is one wherein less than about 10% and preferably less than about 5% of the fusion protein is present as an aggregate in the formulation. An increase in aggregate formation following lyophilization and storage of the lyophilized formulation can be determined. For example, a “stable” lyophilized formulation may be one wherein the increase in aggregate in the lyophilized formulation is less than about 5% or less than about 3%, when the lyophilized formulation is incubated at 40° C. for at least one week. Stability of the fusion protein formulation may be measured using a biological activity assay such as a binding assay as described herein.

A “reconstituted” formulation is one which has been prepared by dissolving a lyophilized fusion protein formulation in a diluent such that the fusion protein is dispersed and/or dissolved in the reconstituted formulation. The reconstituted formulation may be suitable for administration (e.g. parenteral administration) to a patient to be treated with the fusion protein and, may be one which is suitable for subcutaneous administration.

By “isotonic,” it is meant that the formulation of interest has an osmotic pressure from about 240 to about 340 mOsm/kg. In an embodiment, an isotonic formulation is one having an osmotic pressure that is essentially the same as human blood (285-310 mOsm/kg). Isotonicity can be measured using a vapor pressure or a freezing point depression type osmometer.

A “lyoprotectant” is a molecule which, when combined with a protein, significantly prevents or reduces chemical and/or physical instability of the protein upon lyophilization and subsequent storage. Exemplary lyoprotectants include sugars such as sucrose or trehalose; a polyol such as sugar alcohols, e.g. erythritol, arabitol, xylitol, sorbitol, and mannitol; or combinations thereof. In an embodiment, the lyoprotectant may comprise a sugar. In another embodiment, the lyoprotectant may comprise a non-reducing sugar. In a further embodiment, the lyoprotectant may comprise a non-reducing sugar such as sucrose. The lyoprotectant may be added to a pre-lyophilized formulation in a “lyoprotecting amount” which means that, following lyophilization of the protein in the presence of the lyoprotecting amount of the lyoprotectant, the protein essentially retains its physical and chemical stability and biological activity upon lyophilization and storage.

A “diluent” for a lyophilized formulation herein is one which is pharmaceutically acceptable (safe and non-toxic for administration to a human) and is useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution. In an embodiment, the diluent provides a reconstituted formulation suitable for injection. In another embodiment, where the diluent provides a reconstituted formulation suitable for injection, the diluent comprises water for injection (WFI).

A “preservative” for a reconstituted formulation is a compound which can be added to the diluent or to the reconstituted formulation to essentially reduce bacterial action in the reconstituted formulation. In an embodiment, the amount of preservative added is an amount useful to facilitate the production of a multi-use reconstituted formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, allyl parabens such as methyl or propyl paraben, catechol, resorcinol, cyclohexanol, 3-pentanol, and m-cresol.

A “bulking agent” for a lyophilized formulation is a compound which adds mass to the lyophilized mixture and contributes to the physical structure of the lyophilized cake (e.g. facilitates the production of an essentially uniform lyophilized cake which maintains an open pore structure). Exemplary bulking agents include, but are not limited to, mannitol, glycine, and xorbitol.

### Immunoglobulin Fusion Proteins Having Reduced Inflammatory Response

Embodiments of the present invention comprise immunoglobulin fusion proteins having reduced inflammatory response, as well as methods of making such fusion proteins, and methods of use of such fusion proteins.

All immunoglobulins have a hinge region located C terminal to the C_{H}1 domain and separating the antigen-binding end (Fab) and the effector binding (Fc) regions of the molecule. Generally, the hinge domain includes a high preponderance of amino acids such as proline, serine, and threonine, which tend to not form secondary structure. The hinge region may also include cysteine residues which enable the formation of disulfide bonds to link heavy chains to each other.

The immunoglobulin Fc portion may confer several attributes to a fusion protein. For example, addition of the Fc portion of an immunoglobulin polypeptide to a protein of interest may increase the serum half-life of the protein of interest, often from hours to several days. The increase in pharmacokinetic stability is generally a result of the interaction of the linker between C_{H}2 and C_{H}3 regions of the Fc fragment with the FcRn receptor (Wines et al., J. Immunol., 164:5313-5318 (2000)). However, a primary proinflammatory site on immunoglobulin Fc fragments can reside on the hinge region between the C_{H}1 and C_{H}2. This hinge region can interact with FcR1-3 on various leukocytes and trigger these cells to attack the target. (Wines et al., J. Immunol., 164:5313-5318 (2000)).

Thus, although fusion proteins comprising an immunoglobulin Fc polypeptide may provide the advantage of increased stability, immunoglobulin fusion proteins may elicit an inflammatory response when introduced into a host. The inflammatory response may be due, in large part, to the hinge portion of the Fc immunoglobulin of the fusion protein. The proinflammatory response may be a desirable feature if the target of the fusion protein is expressed on a diseased cell type that needs to be eliminated (e.g., a cancer cell, or a population of lymphocytes causing an autoimmune disease). The proinflammatory response may be a neutral feature if the target is a soluble protein, as most soluble proteins do not activate immunoglobulins. However, the proinflammatory response may be a negative feature if the target is expressed on cell types whose destruction would lead to untoward side-effects. Also, the proinflammatory response may be a negative feature if an inflammatory cascade is established at the site of a fusion protein binding to a tissue target, since many mediators of inflammation may be detrimental to surrounding tissue, and/or may cause systemic effects.

The present invention may be embodied in a variety of ways. In certain embodiments, the fusion protein may comprise a non-immunoglobulin polypeptide linked to an immunoglobulin polypeptide or a portion thereof from which the hinge sequence, or a portion thereof, has been removed. As the hinge region of the immunoglobulin chain may be proinflammatory in vivo, embodiments of the fusion proteins of the present invention substitute an amino acid sequence from a non-immunoglobulin polypeptide for the hinge region of the immunoglobulin. In certain embodiments, the non-immunoglobulin polypeptide may comprise a region, as for example, an interdomain linker, which replaces the immunoglobulin hinge region in a manner that allows for correct assembly of the immunoglobulin polypeptide.

For example, in one embodiment, the present invention comprises a fusion protein comprising: (a) an immunoglobulin polypeptide from which the hinge region has been removed, and (b) a non-immunoglobulin polypeptide comprising an uninterrupted portion of an immunoglobulin supergene protein, wherein the uninterrupted portion of the immunoglobulin supergene protein comprises a first non-immunoglobulin polypeptide domain directly linked to the N-terminal end of a first interdomain linker, and wherein the immunoglobulin polypeptide from which the hinge sequences have been removed is operably linked to the C-terminal end of the first interdomain linker. In an embodiment, the immunoglobulin polypeptide is directly linked to the C-terminal end of the first interdomain linker.

The immunoglobulin polypeptide and the non-immunoglobulin polypeptide may be from the same species, or from different species. In an embodiment, the immunoglobulin polypeptide and the non-immunoglobulin polypeptide are from the same species. For example, in one embodiment, both the immunoglobulin polypeptide and the human non-immunoglobulin polypeptide are derived from human proteins.

In some embodiments, the non-immunoglobulin polypeptide domain may comprise a ligand binding domain or ligand binding site, or a portion of a ligand binding domain or ligand binding site. In certain embodiments, the immunoglobulin polypeptide domains may comprise an uninterrupted portion of a ligand binding domain or an uninterrupted portion of ligand binding site.

In certain embodiments, effective removal of a hinge region is determined based on a biological activity that is altered upon removal of the hinge. An example of biological activity that may be altered includes reduction of the ability of the fusion protein to bind to some Fc receptors (e.g., as compared to the immunoglobulin from which the fusion protein is derived) as for example, deletion of the hinge region as described herein. In an embodiment, the interdomain linker sequence from the non-immunoglobulin polypeptide does not promote binding of the fusion protein to a Fc receptor (FcR) at physiological or therapeutically effective concentrations of the fusion protein, or as compared to other immunoglobulins or immunoglobulin fusion proteins. For example, in alternate embodiments, replacement of an immunoglobulin hinge region with an interdomain linker from the non-immunoglobulin protein may reduce binding of the fusion protein to the Fc receptor by at least 2-fold, or 5-fold, or 10-fold, or 20-fold, or 50-fold, or 100-fold, or 500-fold, or 1.000-fold, or 10.000-fold or greater as compared to an immunoglobulin polypeptide having the hinge region. The physiological or therapeutically effective concentration of the fusion protein may vary depending upon the specific nature of the construct. For example, where the fusion protein comprises a non-immunoglobulin portion that binds ligands for a receptor, a therapeutically effective amount may comprise an amount of the fusion protein that can bind to, or compete for, natural receptor ligands.

Also in some embodiments, the fusion proteins of the present invention may induce less inflammation when administered to a subject as compared to fusion proteins having functional Fc hinge sequences. For example, in alternate embodiments, replacement of an immunoglobulin hinge sequence with an interdomain linker from the non-immunoglobulin protein may reduce the ability of the fusion protein to induce inflammation as compared to an immunoglobulin polypeptide having the hinge region.

As described herein, in certain embodiments, the heavy chain immunoglobulin polypeptide may be altered such that the region of the immunoglobulin polypeptide that encodes for a hinge-type region is removed. The Leu-Leu-Gly-Gly (i.e., LLGG) motif of the lower hinge of the Fc domain (Edelman, G. M. et al., Proc. Natl. Acad. Sci. USA 63:78-85 (1969)) (e.g., amino acids 7-10 of SEQ ID NO: 11 and amino acids 19-22 of SEQ ID NO: 24 herein) may be an important Fc-FcR binding sequence (see, e.g., Hutchins, J. T. et al., Proc. Natl. Acad. Sci. USA 92:11980-11984 (1995)). As used herein, for human IgG1, the hinge region is SEQ ID NO: 24 (EPKSCDKTHTCPPCPAPELLGG), with the upper hinge being SEQ ID NO: 25 (EPKSCDKTHT), the middle hinge being SEQ ID NO: 26 (CPPCP), and the lower hinge being SEQ ID NO: 27 (APELLGG). Or, sequences similar to these sequences may function as hinge sequences. Immunoglobulin IgG1 sequences that have a deleted lower hinge region comprising the LLGG Fc receptor domain include SEQ ID NO: 8 and SEQ ID NO: 14 as shown herein. For other immunoglobulins, corresponding hinge sequences are known, and function in an analogous manner (see e.g., Ellison and Hood, 1981, Proc. Natl. Acad. Sci., USA, 79:1984-1988 for the sequence of IgG2). The hinge regions or portions thereof (e.g., an Fc receptor binding region) of other immunoglobulins (i.e., other IgGs, IgAs, IgMs, and the like) may be removed and or modified as well, based on the known sequences for these immunoglobulins and their polypeptide domains.

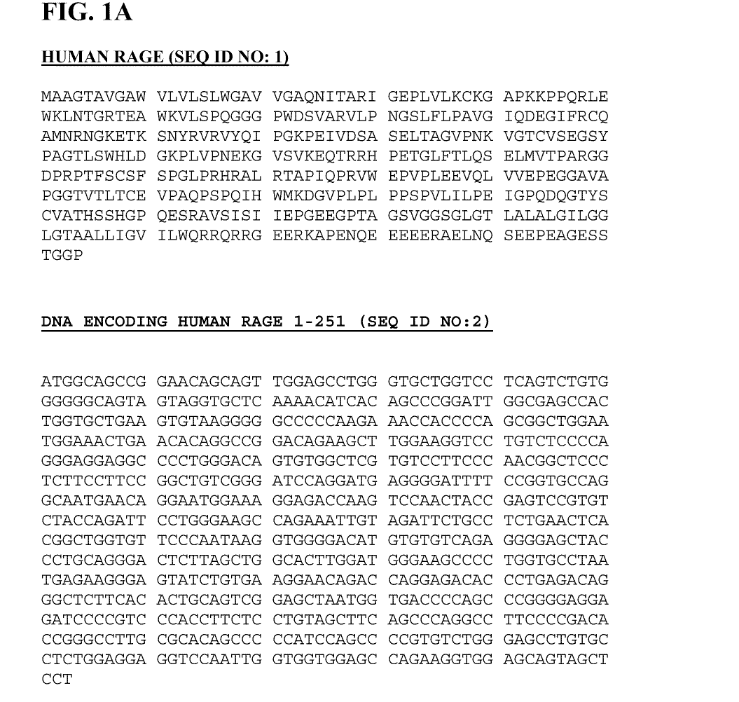

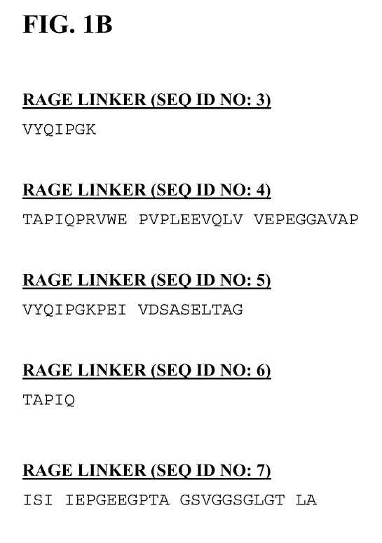

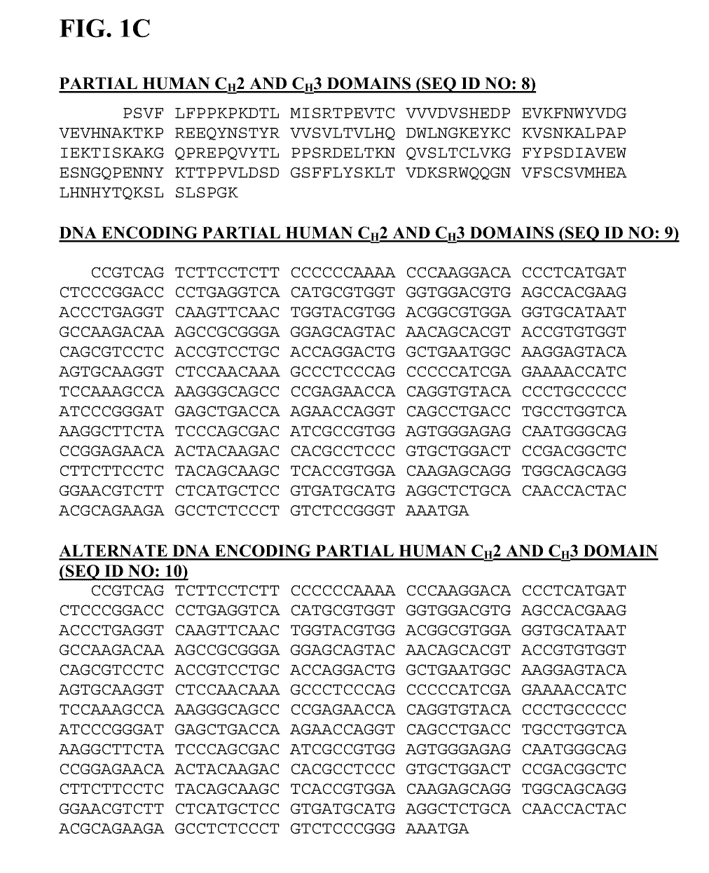

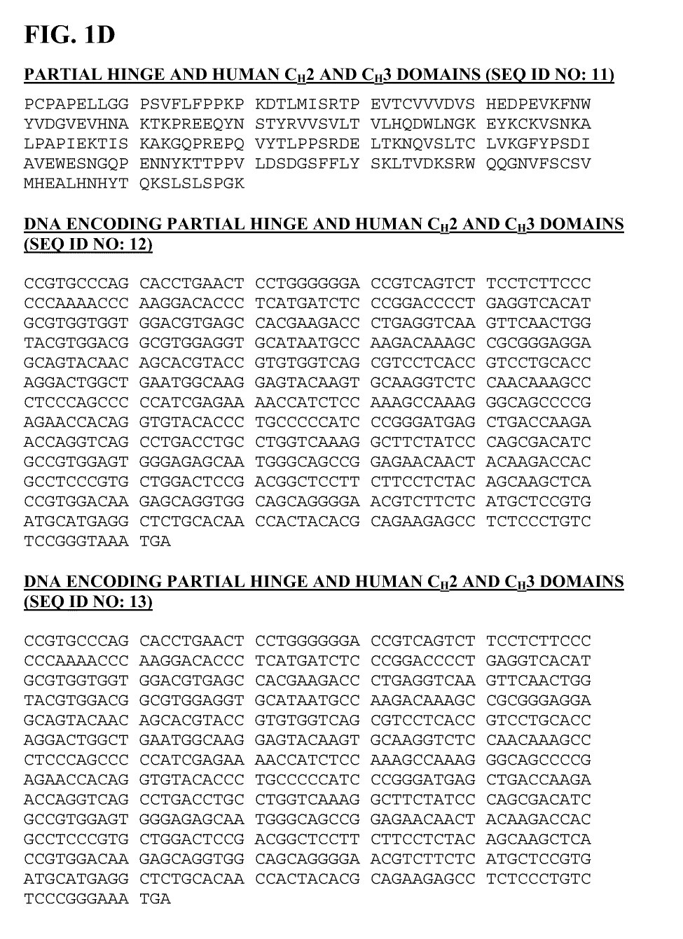

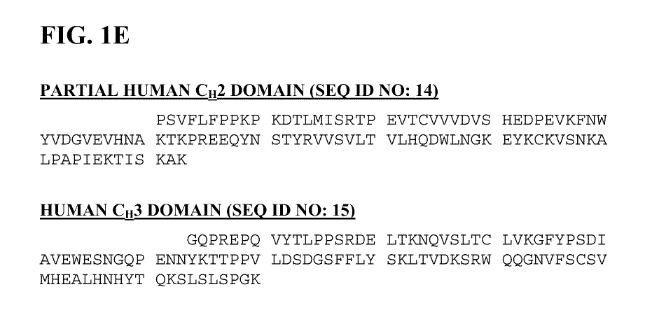

In an embodiment, the immunoglobulin polypeptide comprises at least one constant domain. In certain embodiments, the immunoglobulin polypeptide may comprise at least one of the C_{H}2 domain or the C_{H}3 domain of an immunoglobulin, or a portion of at least one of the C_{H}2 domain or the C_{H}3 domain of an immunoglobulin. In yet other embodiments, the C_{H}1 domain, or a portion thereof may be used. In certain embodiments, the immunoglobulin portion of the fusion protein may comprise an amino acid sequence for the C_{H}2 domain as set forth in SEQ ID NO: 8 or SEQ ID NO: 14, or sequences that are at least 70%, 75%, 80%, 85%, 90%, 95%, 96%, 97%, 98%, or 99% identical thereto from which the lower hinge has been removed (FIG. 1), or sequences at least 90% identical thereto.

For example, in certain embodiments, the fusion protein may comprise an immunoglobulin polypeptide from which the hinge region has been removed, and a non-immunoglobulin polypeptide. The non-immunoglobulin polypeptide may comprise an uninterrupted polypeptide from a non-immunoglobulin protein. In an embodiment, the non-immunoglobulin polypeptide may comprise a first non-immunoglobulin polypeptide domain. Also, the non-immunoglobulin polypeptide may comprise a first interdomain linker.

In certain embodiments, the first non-immunoglobulin polypeptide domain and the first interdomain linker may be derived from a protein that is a member of the immunoglobulin supergene family. Also, in certain embodiments, the first interdomain linker is directly linked to the first non-immunoglobulin polypeptide domain in the protein from which the first non-immunoglobulin polypeptide and first interdomain linker are both derived.

The interdomain linker may be a portion of the non-immunoglobulin protein that is naturally downstream (i.e., found C-terminal to the domain of interest in the native or wild-type protein) or naturally upstream (i.e., found N-terminal to the domain of interest in the native or wild-type protein) of the first non-immunoglobulin polypeptide domain. Thus, in certain embodiments, the fusion proteins comprise a polypeptide domain derived from a non-immunoglobulin protein having the portion of the protein that is naturally downstream or upstream of the domain still attached to the domain. In certain embodiments, the fusion protein comprises a non-immunoglobulin polypeptide domain and a linker that is naturally found downstream (i.e., C-terminal) of the non-immunoglobulin domain. In other embodiments, the fusion protein comprises an non-immunoglobulin polypeptide domain and an interdomain linker that is naturally found upstream (i.e., N-terminal) of the non-immunoglobulin domain. Or, the fusion protein may comprises multiple non-immunoglobulin linkers and domains linked to the immunoglobulin domain from which the hinge sequences have been removed.

The non-immunoglobulin polypeptide may be upstream (N-terminal) or downstream (C-terminal) of the immunoglobulin polypeptide. Also, the non-immunoglobulin domain may be upstream or downstream of the interdomain linker that replaces the hinge domain. In one embodiment, a first interdomain linker is positioned between a first non-immunoglobulin domain and the immunoglobulin portion of the fusion protein. For example, in an embodiment, the N-terminal amino acid of a first interdomain linker is directly linked to the C-terminal amino acid of a first non-immunoglobulin domain and the C-terminal amino acid of the first interdomain linker is operably linked to the N-terminal amino acid of an immunoglobulin domain. For example, the C-terminal amino acid of the first interdomain linker may be directly linked to the N-terminal amino acid of a polypeptide comprising a C_{H}2 domain of an immunoglobulin, or a portion thereof. In an embodiment, the first interdomain linker replaces the Fc hinge linker of the immunoglobulin. In certain embodiments, the first interdomain linker is positioned to replace the immunoglobulin Fc hinge region to allow for correct assembly of the immunoglobulin polypeptide domain. Also in certain embodiments, the first interdomain linker linked to the first non-immunoglobulin domain does not promote binding of the fusion protein to a Fc receptor (FcR) at a therapeutically effective concentration of the fusion protein. In yet other embodiments, the fusion protein may comprise non-immunoglobulin domains that are positioned both N-terminal and C-terminal to an immunoglobulin domain, or immunoglobulin domains that are positioned both N-terminal and C-terminal to a non-immunoglobulin domain.

In yet another embodiment, the non-immunoglobulin polypeptide may comprise a plurality of domains. In some embodiments, the plurality of domains may be linked to each other by an interdomain linker(s). Thus, in certain embodiments, the fusion protein of the present invention may comprise multiple non-immunoglobulin polypeptide domains linked to each other by one or more interdomain linkers. For example, in some embodiments, the non-immunoglobulin polypeptide further comprises a second non-immunoglobulin polypeptide domain that is operably linked to the first non-immunoglobulin polypeptide domain. The second domain may be positioned upstream or downstream to the first non-immunoglobulin domain. In an embodiment, the non-immunoglobulin polypeptide further comprises a second non-immunoglobulin polypeptide domain that is operably linked to the N-terminal end of the first non-immunoglobulin polypeptide domain. In certain embodiments, the second non-immunoglobulin polypeptide domain is directly linked to the first polypeptide domain (i.e., without an interdomain linker). In other embodiments, there may be a second interdomain linker between the first and second non-immunoglobulin polypeptide domains. In some embodiments, the first and second interdomain linkers have different amino acid sequences. In an embodiment, the second non-immunoglobulin polypeptide domain is positioned distal to the immunoglobulin portion of the fusion protein as compared to the first non-immunoglobulin polypeptide domain. Thus, if the non-immunoglobulin polypeptide domain(s) are upstream of the immunoglobulin portion of the fusion protein, the second non-immunoglobulin domain (and any other additional non-immunoglobulin domains) may be positioned upstream of the first non-immunoglobulin domain and first interdomain linker. Alternatively, if the non-immunoglobulin polypeptide domain(s) are downstream of the immunoglobulin portion of the fusion protein, the second non-immunoglobulin domain (and any other additional non-immunoglobulin domains) may be positioned downstream of the first non-immunoglobulin domain and first interdomain linker. In yet other embodiments, the fusion protein may comprise non-immunoglobulin domains that are positioned both N-terminal and C-terminal to an immunoglobulin domain, or immunoglobulin domains that are positioned both N-terminal and C-terminal to a non-immunoglobulin domain.

A variety of polypeptides and/or proteins may be used as the non-immunoglobulin polypeptide of the present invention. Example proteins and polypeptides that may be used include receptors, polypeptide ligands, hormones, cytokines, chemokines, secreted enzymes, or extracellular portions of a transmembrane protein.

In some embodiments, the first non-immunoglobulin polypeptide is a member of the immunoglobulin supergene family, but is not derived from an immunoglobulin or a fragment thereof. In an embodiment, the first non-immunoglobulin protein may comprise the Receptor for Advanced Glycation Endproducts (RAGE), as well as functional variants or isoforms of RAGE, or a portion thereof. RAGE is a member of the immunoglobulin supergene family of molecules. The extracellular (N-terminal) domain of RAGE includes three immunoglobulin-like regions: one V (variable) type domain followed by two C-type (constant) domains (Neeper et al., J. Biol. Chem., 267:14998-15004 (1992); Schmidt et al., Circ. (Suppl.) 96#194 (1997)). A single transmembrane spanning domain and a short, highly charged cytosolic tail follow the extracellular domain. The N-terminal, extracellular domain can be isolated by proteolysis of RAGE or by molecular biological approaches to generate soluble RAGE (sRAGE) comprised of the V and C domains.

Other members of the immunoglobulin supergene family that do not function as prototypical immunoglobulins may also be used. For example, such proteins include T cell receptors, Killer-cell immunoglobulin-like receptors (KIR), proteins that comprise the class I or class II major histocompatibility complex (MHC class I), CD2, the CD3 receptor (γ, δ and ε chains), CD4, CD8, CD28, CD79a and CD79b, CD80 and CD86 (i.e., B7.1 and B7.2), CD147, LFA, CTLA4, certain Fc receptors, Intercellular adhesion molecules (ICAMs), Vascular cell adhesion molecule-1 (VCAM-1), Neural Cell Adhesion Molecule (NCAM), Interleukin-1 receptor type I, Platelet-derived growth factor receptor (PDGFR), and Thymocyte differentiation antigen-1 (i.e., Thy-1 or CD90), as well as functional variants or isoforms of such proteins. In alternate embodiments, the first non-immunoglobulin polypeptide domain and the first interdomain linker comprise an uninterrupted portion of a polypeptide derived from at least one of RAGE, LFA-3, CTLA, or CD4.

In an embodiment where RAGE is used as the non-immunoglobulin polypeptide, the RAGE polypeptide used in the RAGE fusion proteins of the present invention may comprise a fragment of full length RAGE. Thus, in alternate embodiments, the fusion protein may comprise various portions of RAGE.

In some embodiments, the non-immunoglobulin polypeptide may be a protein that has a signal sequence. In other embodiments, the non-immunoglobulin polypeptide may comprise a processed or mature polypeptide that has a signal sequence removed. For example, the signal sequence of RAGE may comprise either residues 1-22 or residues 1-23 of full length RAGE. Thus, in an embodiment where RAGE or a fragment thereof is used as the non-immunoglobulin polypeptide, the RAGE portion of the fusion protein may have the signal sequence removed.

Where RAGE is used as the non-immunoglobulin polypeptide, the RAGE protein may comprise a RAGE V domain or a portion thereof such as the RAGE ligand binding domain (e.g., amino acids 1-118, 23-118, 24-118, 31-118, 1-116, 23-116, 24-116, 31-116, 1-114, 23-114, 24-114, 31-114, 1-54, 23-54, 24-54, 31-54, 1-53, 23-53, 24-53, or 31-53, or fragments thereof, of SEQ ID NO: 1) (FIG. 1). Or fragments of the polypeptides that functionally bind a RAGE ligand may be used. Or, a sequence at least 70%, 75%, 80%, 85%, 90%, 95%, 97%, 98% or 99% identical to the RAGE V domain or a fragment thereof (e.g., as described above) may be used. Further, as is known in the art, in embodiments where the N-terminus of the fusion protein is glutamine, as for example upon removal of the signal sequence comprising residues 1-23 of SEQ ID NO: 1 (e.g., Q24 for a polypeptide comprise amino acids 24-118 or SEQ ID NO: 1), the glutamine may cyclize to form pyroglutamic acid (pE).

Or, for a RAGE fusion protein, larger portions of RAGE may be used. Thus in various embodiments, the RAGE polypeptide may comprise acids 124-221 of human RAGE or a sequence at least 90 percent identical thereto, corresponding to the C1 domain of RAGE. In another embodiment, the RAGE polypeptide may comprise amino acids 227-317 of human RAGE or a sequence at least 90 percent identical thereto, corresponding to the C2 domain of RAGE. Or, the RAGE polypeptide may comprise amino acids 23-123 of human RAGE or a sequence at least 90 percent identical thereto, or amino acids 24-123 of human RAGE or a sequence at least 90 percent identical thereto, corresponding to the V domain of RAGE and a downstream interdomain linker. Or, the RAGE polypeptide may comprise amino acids 24-123 of human RAGE where Q24 cyclizes to form pE or a sequence at least 90 percent identical thereto. Or, the RAGE polypeptide may comprise amino acids 23-226 of human RAGE or a sequence at least 90 percent identical thereto, or amino acids 24-226 of human RAGE or a sequence at least 90 percent identical thereto, corresponding to the V-domain, the C1 domain and the interdomain linker linking these two domains. Or, the RAGE polypeptide may comprise amino acids 24-226 of human RAGE where Q24 cyclizes to form pE, or a sequence at least 90 percent identical thereto.

The interdomain linker may be a peptide that is linked to the non-immunoglobulin domain in the protein from which the non-immunoglobulin domain is derived. In an embodiment where the non-immunoglobulin domain is derived from RAGE, the interdomain linker that replaces the immunoglobulin hinge sequence is derived from RAGE. Thus, in an embodiment, the RAGE polypeptide may comprise a polypeptide comprising a region of human RAGE that is able to substitute for the Fc hinge region. Non-limiting example embodiments of interdomain linkers are depicted in SEQ ID NOs: 3-7 (or a sequence that is at least 70, 75, 80, 85, 90, 95, 97, 98, or 99 percent identical thereto). Or, fragments of these sequences may be used.

In one embodiment, the immunoglobulin polypeptide may comprise an immunoglobulin heavy chain or a portion (i.e., fragment) thereof. For example, the heavy chain fragment may comprise a polypeptide derived from the Fc fragment of an immunoglobulin but that does not include the Fc hinge region. Thus, in an embodiment, the immunoglobulin may comprise at least one of the C_{H}2 and C_{H}3 domains of the immunoglobulin heavy chain (or a fragment thereof) as a monomer. In certain embodiments, the immunoglobulin polypeptide may comprise an Fc fragment without the hinge region. For example, in some embodiments, the immunoglobulin polypeptide may comprise a portion of the C_{H}2 domain and at least a portion of the C_{H}3 domain of an immunoglobulin. The portion of the C_{H}2 domain may comprise the C_{H}2 domain with the hinge region removed. In one embodiment, the immunoglobulin polypeptide comprises a portion of the C_{H}2 domain and the C_{H}3 domain of an immunoglobulin. The heavy chain (or portion thereof) may be derived from any one of the known heavy chain isotypes: IgG (γ), IgM (μ), IgD (δ), IgE (ε), or IgA (α). In addition, the heavy chain (or portion thereof) may be derived from any one of the known heavy chain subtypes: IgG1 (γ1), IgG2 (γ2), IgG3 (γ3), IgG4 (γ4), IgA1 (α1), IgA2 (α2), or mutations of these isotypes or subtypes that alter the biological activity. As an example embodiments, the polypeptide comprising the C_{H}2 and C_{H}3 domains of a human IgG1 or a portion thereof may comprise SEQ ID NO: 8. The immunoglobulin peptide may be encoded by the nucleic acid sequence of SEQ ID NO: 9. The immunoglobulin sequence in SEQ ID NO: 8 may also be encoded by SEQ ID NO: 10, where silent base changes for the codons that encode for proline (CCG to CCC) and glycine (GGT to GGG) at the C-terminus of the sequence remove a cryptic RNA splice site near the terminal codon.

As recognized in the art, a C_{H}3 domain of a fusion protein of the present invention may have its C-terminal amino acid cleaved off via post-translational modification when expressed in certain recombinant systems. (See e.g, Li, et al., BioProcessing J., 2005; 4, 23-30). In an embodiment, the C-terminal amino acid cleaved off is lysine (K). Thus, in an embodiment, the immunoglobulin portion of the fusion has the amino acid sequence as set forth in SEQ ID NO: 8 without the C-terminal lysine (K).

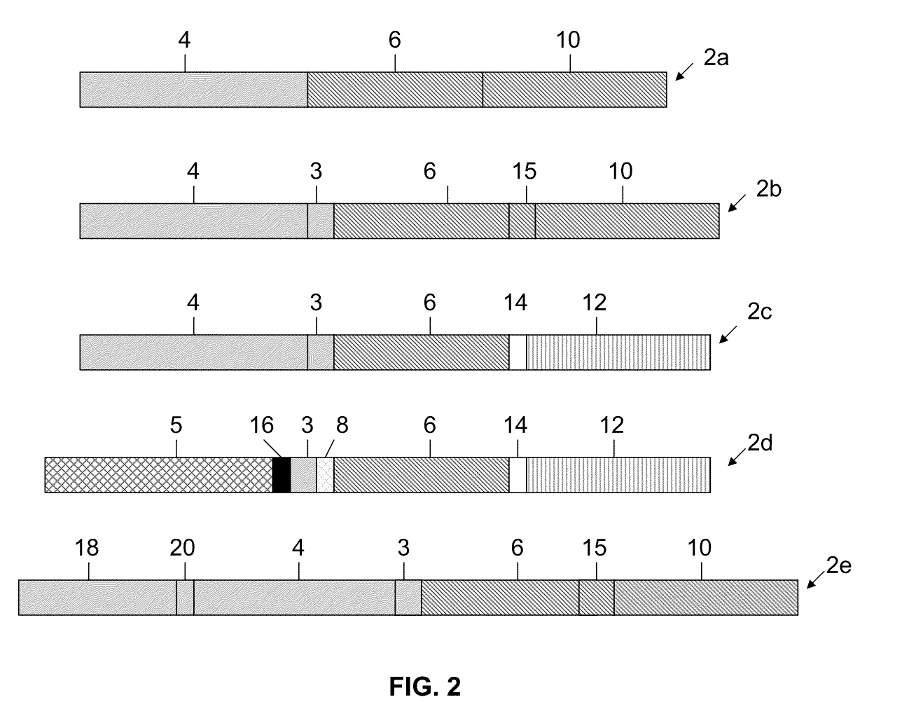

FIG. 2 shows a schematic representation of several non-limiting embodiments of the fusion proteins of the present invention. As depicted in FIG. 2, polypeptides that are derived from the same molecule have the same shading. In one embodiment (2a), the fusion protein may comprise a first, non-immunoglobulin polypeptide domain (4) directly linked to an immunoglobulin polypeptide, or a fragment thereof (6). Thus, in certain embodiments, the portion of the non-immunoglobulin polypeptide (4) that replaces the hinge domain of the immunoglobulin polypeptide is part of a domain of a non-immunoglobulin polypeptide or protein.

In an embodiment, the immunoglobulin polypeptide may comprise two immunoglobulin domains (6, 10) or fragments thereof. For example, in an embodiment, one domain (e.g., 6) is the C_{H}2 domain and the other domain (e.g., 10) is the C_{H}3 domain. In certain embodiments, the C_{H}2 domain is truncated at the N-terminus to remove the hinge domain or a portion thereof.

In an embodiment (e.g., 2b), the non-immunoglobulin polypeptide (4) may comprise an interdomain linker that replaces the immunoglobulin Fc hinge domain. The non-immunoglobulin polypeptide (4) and/or interdomain linker (3) may be directly linked to the immunoglobulin polypeptide (6) or polypeptides (6, 10) (e.g., construct 2b or 2c). Or, the interdomain linker (3) replacing the hinge may be linked to the immunoglobulin polypeptide (6) or polypeptides (6, 10) by a second interdomain linker peptide (8) (e.g., construct 2d). Also, in some embodiments, there may be a second linker sequence (16) between a functional domain (e.g., 5) of the non-immunoglobulin polypeptide and the interdomain linker (3) that replaces the hinge region of the immunoglobulin (e.g., construct 2d).

The immunoglobulin polypeptide may comprise two immunoglobulin domains from the same immunoglobulin (e.g., 6, 10) or may be a chimeric polypeptide, comprising immunoglobulin domains (e.g., 6, 12) from two different immunoglobulins (e.g., construct 2c). In alternate embodiments, the immunoglobulin domains may be directly linked (e.g., 2a), or there may be a linker or another polypeptide domain (14, 15) separating the two domains (e.g., 2b, 2c, 2d and 2e). For example, in an embodiment, the linker between the two immunoglobulin domains comprises a FcRn binding region found between the C_{H}2 and C_{H}3 domains. The linker may be from the same polypeptides as at least one of the immunoglobulin domains (e.g., 15), or may be from a different polypeptide (e.g., 14).

In an embodiment, the interdomain linker (3) that replaces the hinge region of Fc and the non-immunoglobulin polypeptide (4) are derived from the same molecule (e.g., constructs 2b, 2c and 2e). In another embodiment, the polypeptide that replaces the hinge region of Fc (3) and the non-immunoglobulin polypeptide (5) are derived from different molecules (e.g., construct 2d). In yet another embodiment, there are a plurality of non-immunoglobulin polypeptides which may or may not be from the same molecule as each other and/or the polypeptide (3) that replaces the immunoglobulin Fc domain (e.g., construct 2e).

In one embodiment, the polypeptide (3) that replaces the hinge sequence is linked to a first domain from the non-immunoglobulin protein (4) such that the C-terminal amino acid of the non-immunoglobulin domain (4) is directly linked to the N-terminal amino acid of the sequence (3) that replaces the hinge domain. In an embodiment, this positioning is the same as is found in the protein from which (4) and (3) are derived. Also, in an embodiment, the C-terminal amino acid of the sequence that replaces the hinge domain (3) is directly linked to the N-terminal amino acid of a polypeptide comprising a C_{H}2 domain of an immunoglobulin, or a fragment thereof (6) from which the hinge region has been removed. The polypeptide comprising a C_{H}2 domain of an immunoglobulin may comprise at least one of the C_{H}2 and C_{H}3 domains of a human IgG1 or a portion of either, or both, of these domains. As an example embodiment, the polypeptide comprising the C_{H}2 and C_{H}3 domains of a human IgG1, or a portion thereof from which the hinge region has been removed may comprise SEQ ID NO: 8.

As shown in FIG. 2, in some embodiments, the fusion protein may comprise one, two, or more domains derived from a non-immunoglobulin protein and two immunoglobulin domains derived from a human Fc polypeptide. The fusion protein may comprise a first functional domain (4) and a first interdomain linker (3) linked to a second functional domain (18) and a second interdomain linker (20), such that the N-terminal amino acid of the second interdomain linker (20) is linked to the C-terminal amino acid of the second domain (18), the N-terminal amino acid of the first domain (4) is linked to C-terminal amino acid of the second interdomain linker (20), the N-terminal amino acid of the hinge-replacing sequence (3) is linked to C-terminal amino acid of the first domain (4), and the C-terminal amino acid of the hinge-replacing domain (3) is directly linked to the N-terminal amino acid of the C_{H}2 immunoglobulin domain with the hinge sequences removed (6) (e.g., construct 2e).

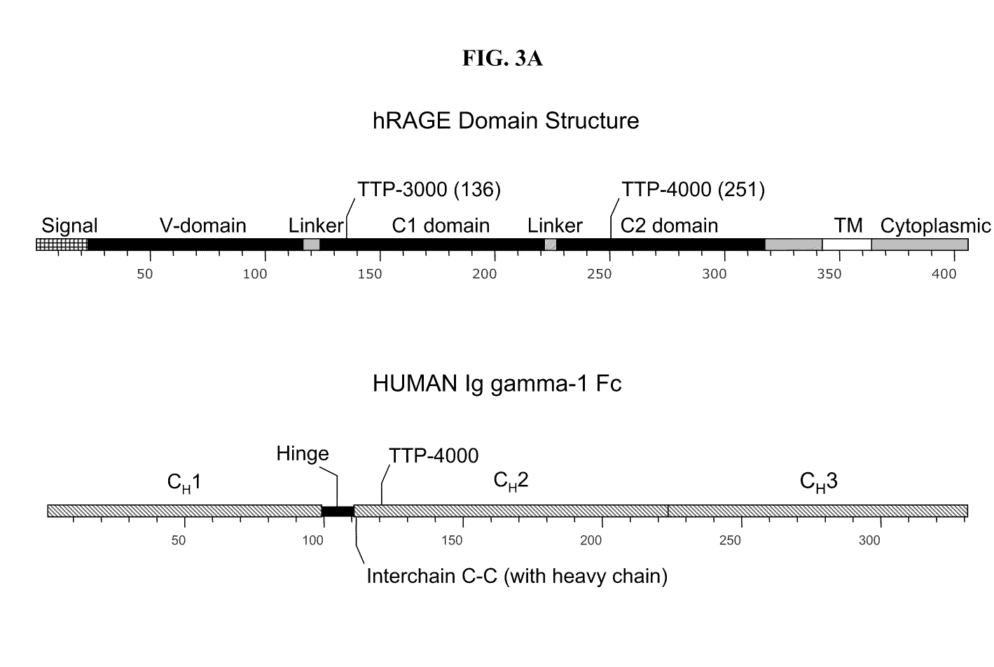

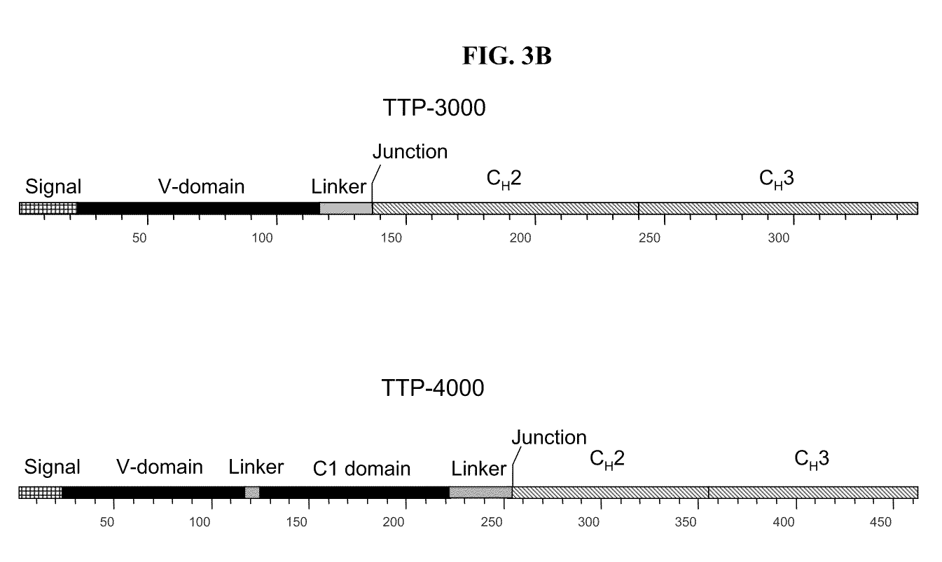

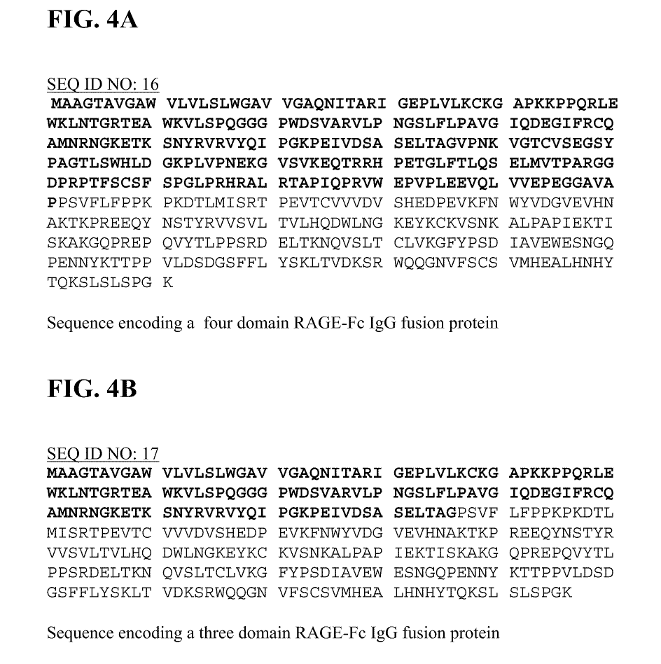

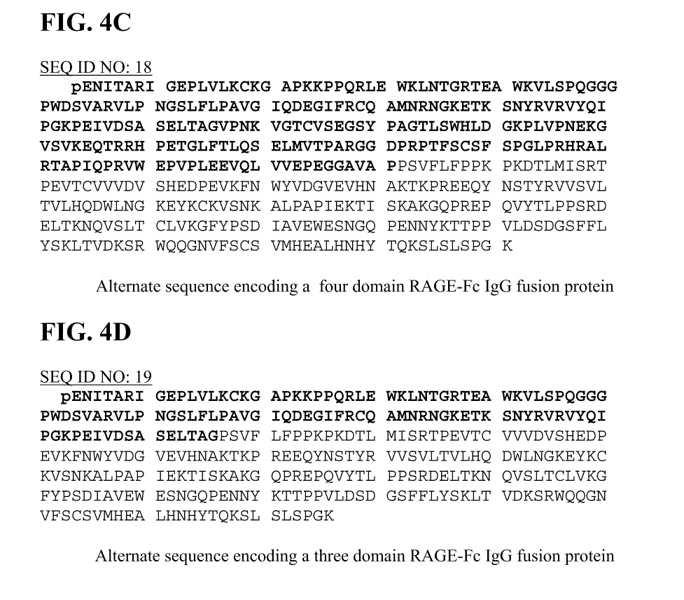

For example, in one embodiment, the fusion protein may comprise two domains from RAGE (the RAGE V-domain and RAGE C1 domain) with the sequence that replaces the immunoglobulin hinge being derived from the portion of RAGE that is downstream of the C1 domain. Or, the fusion protein may comprise one domain from RAGE (the V-domain) with the sequence that replaces the immunoglobulin hinge derived from the portion of RAGE that is downstream of the V domain. FIG. 3 shows a schematic comparison of RAGE and IgG1. Thus, as shown in FIG. 3A RAGE includes a V domain, C1 domain, and C2 domain that are similar in organization to the C_{H}1 domain, C_{H}2 domain, and the C_{H}3 domain of IgG. (FIG. 3A). Thus, in an embodiment, RAGE may be cleaved such that a fusion protein comprises the V domain of RAGE linked to an immunoglobulin polypeptide comprising the C_{H}2 domain of IgG without the hinge sequences and that is linked to the C_{H}3 domain of IgG (e.g., TTP-3000) (FIG. 3B). Or, in an embodiment, RAGE may be cleaved such that a fusion protein comprises the V and C1 domains of RAGE linked to the to an immunoglobulin polypeptide comprising C_{H}2 domain of IgG without the hinge sequences and that is linked to the C_{H}3 domain of IgG (e.g., TTP-4000) (FIG. 3B). In alternate embodiments, the fusion protein comprises SEQ ID NO: 16 or SEQ ID NO: 17, or a fragment thereof (FIG. 4). In alternate embodiments, a RAGE fusion protein of the present invention may be encoded by SEQ ID NOs: 16 or 17 having the C-terminal lysine removed, or SEQ ID NOs: 16 and 17 without a signal sequence (e.g., SEQ ID NO: 18 and 19, respectively) that may, in certain embodiments, have the C-terminal lysine removed (FIG. 4). Or sequences at least 70, 75, 80, 85, 90, 95, 97, 98, or 99 percent identical to these sequences may be used.

An interdomain linker may be a peptide sequence that is naturally downstream of, and thus linked to, a particular domain. In an embodiment, the polypeptide that replaces the immunoglobulin hinge domain is derived from RAGE. For example, for the RAGE V domain, the interdomain linker may comprise amino acid sequences that are naturally downstream from the RAGE V domain. In an embodiment, the linker may comprise SEQ ID NO: 3, corresponding to amino acids 117-123 of full-length RAGE (FIG. 1). Or, the linker may comprise a peptide having additional portions of the natural sequence. For example, an interdomain linker comprising several amino acids (e.g., 1-3, 1-5, or 1-10, or 1-15 amino acids) upstream and downstream of SEQ ID NO: 3 (FIG. 1) may be used. Thus, in one embodiment, an interdomain linker for RAGE comprises SEQ ID NO: 5 (FIG. 1) comprising amino acids 117-136 of full-length RAGE. Or, fragments of SEQ ID NO: 5 deleting, for example, 1, 2, or 3, or 1-3, 1-5, 1-10, 1-15 amino acids from either end of the linker may be used. In alternate embodiments, the linker may comprise a peptide that is at least 70% identical, or 80% identical, or 90% identical to SEQ ID NO: 3 or SEQ ID NO: 5.

For the RAGE C1 domain, the interdomain linker may comprise a peptide sequence that is naturally downstream of the C1 domain. In an embodiment, the interdomain linker may comprise SEQ ID NO: 4 (FIG. 1), corresponding to amino acids 222-251 of full-length RAGE. Or, the interdomain linker may comprise a peptide having additional portions of the natural RAGE sequence. For example, an interdomain linker comprising several (1-3, 1-5, or 1-10, or 1-15 amino acids) amino acids upstream and downstream of SEQ ID NO: 4 may be used. Or, fragments of SEQ ID NO: 4 may be used, deleting for example, 1-3, 1-5, or 1-10, or 1-15 amino acids from either end of the linker. For example, in one embodiment, an interdomain linker may comprise SEQ ID NO: 6 (FIG. 1), corresponding to amino acids 222-226. In alternate embodiments, the linker may comprise a peptide that is at least 70% identical, or 80% identical, or 90% identical to SEQ ID NO: 4.

Or an RAGE interdomain linker may comprise SEQ ID NO: 7 (FIG. 1), corresponding to RAGE amino acids 318-342 downstream of the RAGE C2 domain. Or, the interdomain linker may comprise a peptide comprising several (1-3, 1-5, or 1-10, or 1-15 amino acids) amino acids upstream and downstream of SEQ ID NO: 7 may be used. Or, fragments of SEQ ID NO: 7 may be used, deleting for example, 1-3, 1-5, or 1-10, or 1-15 amino acids from either end of the linker. Or the linker may comprise a peptide that is at least 70% identical, or 80% identical, or 90% identical to SEQ ID NO: 7 or a fragment thereof.

Furthermore, for each of the sequences described herein, in certain embodiments, individual substitutions, deletions or additions which alter, add or delete a single amino acid or a small percentage of amino acids (typically less than about 5%, more typically less than about 1%) in an encoded sequence or conservatively modified variations where the alterations result in the substitution of an amino acid with a chemically similar amino acid may be used. Conservative substitution tables providing functionally similar amino acids are well known in the art. The following example groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” also refers to utilizing a substituting amino acid which is identical to the amino acid being substituted except that a functional group in the side chain is protected with a suitable protecting group.

Also, in certain embodiments of the fusion proteins of the present invention, amino acids may become chemically modified from their natural structure, either by enzymatic or non-enzymatic reaction mechanisms. For example, as described herein, in certain embodiments, an N-terminal glutamic acid or glutamine may cyclize, with loss of water, to form pyroglutamic acid (pyroE or pE) (Chelius et al., Anal. Chem, 78: 2370-2376 (2006) and Burstein et al., Proc. National Acad. Sci., 73:2604-2608 (1976)). Alternatively, a fusion protein having an N-terminal pyroglutamic acid could potentially be accessed through a nucleic acid sequence encoding for glutamic acid at the position in the protein that via post-translational processing becomes the N-terminus (e.g., where residue 24 of SEQ ID NO: 1 is glutamate rather than a glutamine).

### Methods of Producing

The present invention also comprises a method to make a fusion protein. In one embodiment, the method comprises covalently linking a non-immunoglobulin polypeptide to an immunoglobulin polypeptide. Since the hinge portion of the immunoglobulin chain may be proinflammatory in vivo, the fusion protein of the present invention may comprise a non-immunoglobulin sequence that is substituted for the hinge region. For example, in an embodiment, the fusion protein of the present invention may comprise an interdomain linker derived from a non-immunoglobulin polypeptide rather than a hinge polypeptide derived from an immunoglobulin. In certain embodiments, the non-immunoglobulin polypeptide may comprise a region, as for example, an interdomain linker, which replaces the immunoglobulin hinge region in a manner that allows for correct assembly of the immunoglobulin polypeptide. In an embodiment, the interdomain linker sequence from the non-immunoglobulin polypeptide does not promote binding of the fusion protein to a Fc receptor (FcR) at physiological and/or therapeutically effective concentrations of the fusion protein.

For example, in certain embodiments, the present invention comprises a method of making a fusion protein comprising covalently linking: (a) an immunoglobulin polypeptide from which the hinge region has been removed; and (b) a non-immunoglobulin polypeptide comprising an uninterrupted portion of an immunoglobulin supergene protein. wherein the uninterrupted portion of the immunoglobulin supergene protein comprises a first non-immunoglobulin polypeptide domain directly linked to the N-terminal end of a first interdomain linker, and wherein the immunoglobulin polypeptide is operably linked to the C-terminal end of the first interdomain linker.

In an embodiment, the immunoglobulin polypeptide comprises at least one constant domain. In certain embodiments, the immunoglobulin polypeptide may comprise at least one of the C_{H}2 domain or the C_{H}3 domain of an immunoglobulin, or a portion of the C_{H}2 domain or a portion the C_{H}3 domain of an immunoglobulin. In certain embodiments, the immunoglobulin polypeptide may comprise an Fc fragment without the hinge region. For example, in some embodiments, the immunoglobulin polypeptide may comprise a portion of the C_{H}2 domain and at least a portion of the C_{H}3 domain of an immunoglobulin. The portion of the C_{H}2 domain may comprise the C_{H}2 domain with the hinge region removed. In some embodiments, the immunoglobulin polypeptide comprises a portion of the C_{H}2 domain and the C_{H}3 domain of an immunoglobulin. In yet other embodiments, the C_{H}1 domain, or a portion thereof may be used.

In one embodiment, the immunoglobulin comprises a C_{H}2 domain from which the hinge sequences have been removed. For example, for human IgG1 C_{H}2 polypeptide comprises the lower hinge region. The Leu-Leu-Gly-Gly (i.e., LLGG) motif of the lower hinge of the Fc domain (e.g., amino acids 7-10 of SEQ ID NO: 11 and amino acids 19-22 of SEQ ID NO: 24 herein) may be an important Fc-FcR binding sequence. Immunoglobulin sequences that have a deleted lower hinge region comprising the LLGG Fc receptor domain include SEQ ID NO: 8 and SEQ ID NO: 14 as shown herein. Where other immunoglobulin polypeptides are used, the hinge regions of these other immunoglobulins (i.e., other IgGs, IgAs, IgMs, and the like) may be removed as well, based on the known sequences for these immunoglobulins and their polypeptide domains. In certain embodiments, effective removal of a hinge region is determined based on a biological activity that is altered upon removal of the hinge. An example of biological activity that may be altered includes reduction of the ability of the fusion protein to bind to some Fc receptors as for example, by modification or deletion of the hinge region as described herein. In certain embodiments, the immunoglobulin portion of the fusion protein may comprise an amino acid sequence as set forth in SEQ ID NO: 8 or SEQ ID NO: 14, or sequences at least 90% identical thereto.

Thus, in certain embodiments, the fusion protein made by the methods of the present invention may comprise an immunoglobulin polypeptide from which the hinge region has been removed, and a non-immunoglobulin polypeptide. In an embodiment, the non-immunoglobulin polypeptide may comprise a first non-immunoglobulin polypeptide domain. Also, the non-immunoglobulin polypeptide may comprise a first interdomain linker.

In certain embodiments, the first non-immunoglobulin polypeptide domain and the first interdomain linker may be derived from a protein that is a member of the immunoglobulin supergene family. Also, in certain embodiments, the first interdomain linker may be directly linked to the first non-immunoglobulin polypeptide domain in the protein from which the first non-immunoglobulin polypeptide domain and first interdomain linker are both derived.

In some embodiments, the non-immunoglobulin polypeptide domain may comprise a ligand binding domain or ligand binding site, or a portion of a ligand binding domain or ligand binding site. In certain embodiments, the immunoglobulin polypeptide domains may comprise an uninterrupted portion of a ligand binding domain or an uninterrupted portion of ligand binding site.

In an embodiment, the non-immunoglobulin polypeptide is an uninterrupted non-immunoglobulin polypeptide. For example, the interdomain linker may be a portion of the non-immunoglobulin protein that is naturally downstream (i.e., found C-terminal to the domain of interest in the native or wild-type protein) or naturally upstream (i.e., found N-terminal to the domain of interest in the native or wild-type protein) of the first non-immunoglobulin polypeptide domain. Thus, in certain embodiments, the fusion proteins comprise a polypeptide domain derived from a non-immunoglobulin protein having the portion of the protein that is naturally downstream or upstream of the domain still attached to the non-immunoglobulin domain. In certain embodiments, the fusion protein comprises a non-immunoglobulin polypeptide domain and a linker that is naturally found N-terminal of the non-immunoglobulin domain. In other embodiments, the fusion protein comprises an non-immunoglobulin polypeptide domain and an interdomain linker that is naturally found C-terminal of the non-immunoglobulin domain.

The non-immunoglobulin polypeptide may be upstream (N-terminal) or downstream (C-terminal) of the immunoglobulin polypeptide. Also, the non-immunoglobulin domain may be upstream or downstream of the interdomain linker that replaces the hinge domain. In an embodiment, the first interdomain linker is positioned between the first non-immunoglobulin domain and the immunoglobulin portion of the fusion protein. For example, in an embodiment, the N-terminal amino acid of the first interdomain linker is linked to the C-terminal amino acid of the first non-immunoglobulin domain and the C-terminal amino acid of the first interdomain linker is linked to the N-terminal amino acid of an immunoglobulin domain. For example, the C-terminal amino acid of the first interdomain linker may be directly linked to the N-terminal amino acid of a polypeptide comprising a C_{H}2 domain of an immunoglobulin, or a portion thereof (e.g., the C_{H}2 domain having the hinge sequences removed). In an embodiment, the first interdomain linker replaces the Fc hinge region of the immunoglobulin. In certain embodiments, the first interdomain linker is positioned to replace the immunoglobulin Fc hinge region to allow for correct assembly of the immunoglobulin polypeptide domain.

For example, in an embodiment, the present invention comprises methods to make a fusion protein comprising: covalently linking (a) an immunoglobulin polypeptide from which the hinge region has been removed, and (b) a non-immunoglobulin polypeptide. In an embodiment, the non-immunoglobulin polypeptide comprises an uninterrupted portion of an immunoglobulin supergene protein. In one embodiment, the uninterrupted portion of the immunoglobulin supergene protein may comprise a first non-immunoglobulin polypeptide domain directly linked to the N-terminal end of a first interdomain linker. The fusion protein may also have the immunoglobulin polypeptide operably linked to the C-terminal end of the first interdomain linker. In an embodiment, the immunoglobulin polypeptide is directly linked to the C-terminal end of the first interdomain linker.

The immunoglobulin polypeptide and the non-immunoglobulin polypeptide may be from the same species, or from different species. In an embodiment, the immunoglobulin polypeptide and the non-immunoglobulin polypeptide are from the same species. For example, in an embodiment, both the immunoglobulin polypeptide and the human non-immunoglobulin polypeptide are derived from human proteins.

In yet other embodiments, the non-immunoglobulin polypeptide may comprise a plurality of domains. In some embodiments, the multiple domains may be linked to each other by an interdomain linker(s). Thus, in certain embodiments, the fusion protein of the present invention may comprise multiple non-immunoglobulin polypeptide domains linked to each other by one or more interdomain linkers. For example, in some embodiments, the non-immunoglobulin polypeptide further comprises a second non-immunoglobulin polypeptide domain that is operably linked to the first non-immunoglobulin polypeptide domain. The second domain may be positioned upstream or downstream to the first non-immunoglobulin domain. In an embodiment, the non-immunoglobulin polypeptide further comprises a second non-immunoglobulin polypeptide domain that is operably linked to the N-terminal end of the first non-immunoglobulin polypeptide domain. In certain embodiments, the second domain is directly linked to the first domain. In other embodiments, there may be a second interdomain linker between the first and second non-immunoglobulin polypeptide domains. In some embodiments, the first and second interdomain linkers have different amino acid sequences. In an embodiment, the second non-immunoglobulin polypeptide domain is positioned distal to the immunoglobulin portion of the fusion protein as compared to the first non-immunoglobulin polypeptide domain. Thus, if the non-immunoglobulin domain(s) are upstream of the immunoglobulin portion of the fusion protein, the additional non-immunoglobulin polypeptide domain(s) may be positioned upstream of the first non-immunoglobulin domain and first interdomain linker. Alternatively, if the non-immunoglobulin domain(s) are downstream of the immunoglobulin portion of the fusion protein, the additional non-immunoglobulin domains may be positioned downstream of the first non-immunoglobulin domain and first interdomain linker. In yet other embodiments, the fusion protein may comprise non-immunoglobulin domains that are positioned both N-terminal and C-terminal to an immunoglobulin domain or immunoglobulin domains that are positioned both N-terminal and C-terminal to a non-immunoglobulin domain.

In certain embodiments, the first interdomain linker linked to the first non-immunoglobulin domain does not promote binding of the fusion protein to a Fc receptor (FcR) at a therapeutically effective concentration of the fusion protein. For example, in one embodiment, the first interdomain linker is positioned to replace the immunoglobulin Fc hinge region. For example, in alternate embodiments, replacement of an immunoglobulin hinge region with an interdomain linker from the non-immunoglobulin protein may reduce binding of the fusion protein to the Fc receptor by at least 2-fold, or 5-fold, or 10-fold, or 20-fold, or 50-fold, or 100-fold, or 500-fold, or 1.000-fold, or 10,000 fold or greater as compared to an immunoglobulin polypeptide having the hinge region.

Also in some embodiments, the fusion proteins of the present invention may induce less inflammation when administered to a subject as compared to fusion proteins having functional Fc hinge sequences. For example, in alternate embodiments, replacement of an immunoglobulin hinge region with an interdomain linker from the non-immunoglobulin protein may reduce the ability of the fusion protein to induce inflammation as compared to an immunoglobulin polypeptide having the hinge region.

A variety of polypeptides and/or proteins may be used as the non-immunoglobulin polypeptide of the present invention. Example proteins and polypeptides that may be used include receptors, polypeptide ligands, hormones, cytokines, chemokines, secreted enzymes, or extracellular portions of a transmembrane protein.

In one embodiment, the first non-immunoglobulin polypeptide is a member of the immunoglobulin supergene family, but is not derived from an immunoglobulin or a fragment thereof. Thus, members of the immunoglobulin supergene family that do not function as prototypical immunoglobulins may be used as the non-immunoglobulin polypeptide. Such proteins include the following proteins, or protein variants or isoforms: RAGE, T cell receptors, Killer-cell immunoglobulin-like receptors (KIR), proteins that comprise the class I or class II major histocompatibility complex (MHC class I), CD2, the CD3 receptor (γ, δ and ε chains), CD4, CD8, CD28, CD79a and CD79b, CD80 and CD86 (i.e., B7.1 and B7.2), CD147, LFA, and CTLA4, certain Fc receptors, Intercellular adhesion molecules (ICAMs), Vascular cell adhesion molecule-1 (VCAM-1), Neural Cell Adhesion Molecule (NCAM), Interleukin-1 receptor type I, Platelet-derived growth factor receptor (PDGFR), and Thymocyte differentiation antigen-1 (i.e., Thy-1 or CD90). In alternate embodiments, the first non-immunoglobulin polypeptide domain and the first interdomain linker comprise an uninterrupted portion of a polypeptide derived from at least one of RAGE, LFA-3, CTLA, or CD4.

The fusion protein may be engineered by recombinant DNA techniques. In an embodiment, the fusion protein comprising a non-immunoglobulin polypeptide and the immunoglobulin polypeptide may be encoded by a recombinant DNA construct. In an embodiment, the recombinant DNA construct encodes a fusion protein comprising: (a) an immunoglobulin polypeptide from which the hinge region has been removed, and (b) a non-immunoglobulin polypeptide comprising an uninterrupted portion of an immunoglobulin supergene protein. In some embodiments, the uninterrupted portion of the immunoglobulin supergene protein comprises a first non-immunoglobulin polypeptide domain directly linked to the N-terminal end of a first interdomain linker. The fusion protein may also comprise a structure wherein the immunoglobulin polypeptide is operably linked to the C-terminal end of the first interdomain linker. For example, in one embodiment, the present invention comprises an isolated nucleic acid encoding a fusion protein comprising: (a) an immunoglobulin polypeptide from the hinge region has been removed; and (b) a non-immunoglobulin polypeptide comprising an uninterrupted portion of an immunoglobulin supergene protein, wherein the uninterrupted portion of the immunoglobulin supergene protein comprises a first non-immunoglobulin polypeptide domain directly linked to the N-terminal end of a first interdomain linker, and wherein the immunoglobulin polypeptide is operably linked to the C-terminal end of the first interdomain linker. In an embodiment, the immunoglobulin polypeptide is directly linked to the C-terminal end of the first interdomain linker.

For example, in one embodiment, the present invention may comprise generating an isolated nucleic acid sequence encoding a fusion protein comprising a first non-immunoglobulin polypeptide comprising an interdomain linker region which replaces the immunoglobulin hinge region in a manner that allows for correct assembly of an immunoglobulin polypeptide and that is operably linked to an immunoglobulin polypeptide.

The method may further comprise the step of incorporating the DNA construct into an expression vector. For example, in one embodiment, the present invention comprises an expression vector comprising a nucleic acid encoding: (a) an immunoglobulin polypeptide from which the hinge region has been removed; and (b) a non-immunoglobulin polypeptide comprising an uninterrupted portion of an immunoglobulin supergene protein, wherein the uninterrupted portion of the immunoglobulin supergene protein comprises a first non-immunoglobulin polypeptide domain directly linked to the N-terminal end of a first interdomain linker, and wherein the immunoglobulin polypeptide is operably linked to the C-terminal end of the first interdomain linker. In one embodiment, the immunoglobulin polypeptide is directly linked to the C-terminal end of the first interdomain linker.

Also, the method may comprise the step of inserting the expression vector into a host cell and growing the host cells under conditions such that the fusion protein of the invention is expressed. In some embodiments, the present invention may comprise a cell transfected with an expression vector, such that the cell expresses a fusion protein comprising: (a) an immunoglobulin polypeptide from which the hinge region has been removed; and (b) a non-immunoglobulin polypeptide comprising an uninterrupted portion of an immunoglobulin supergene protein wherein the uninterrupted portion of the immunoglobulin supergene protein comprises a first non-immunoglobulin polypeptide domain directly linked to the N-terminal end of a first interdomain linker, and wherein the immunoglobulin polypeptide is operably and/or directly linked to the C-terminal end of the first interdomain linker. In one embodiment, mammalian cells are used.

The immunoglobulin polypeptide of the fusion protein may comprise a polypeptide derived from an immunoglobulin. The heavy chain (or portion thereof) may be derived from any one of the known heavy chain isotypes: IgG (γ), IgM (μ), IgD (δ), IgE (ε), or IgA (α). In addition, the heavy chain (or portion thereof) may be derived from any one of the known heavy chain subtypes: IgG1 (γ1), IgG2 (γ2), IgG3 (γ3), IgG4 (γ4), IgA1 (α1), IgA2 (α2), or mutations of these isotypes or subtypes that alter the biological activity. The second polypeptide may comprise the C_{H}2 and C_{H}3 domains of a human IgG1 or a portion of either, or both, of these domains.

For example, for human IgG1, the C_{H}2 polypeptide comprises the lower hinge region. The Leu-Leu-Gly-Gly (i.e., LLGG) motif of the lower hinge of the Fc domain (e.g., amino acids 7-10 of SEQ ID NO: 11 and amino acids 19-22 of SEQ ID NO: 24 herein) may be an important Fc-FcR binding sequence. Immunoglobulin sequences that have a deleted lower hinge region comprising the LLGG Fc receptor domain include SEQ ID NO: 8 and SEQ ID NO: 14 as shown herein. The hinge regions of other immunoglobulins (i.e., other IgGs, IgAs, IgMs, and the like) may be removed and as well, based on the known sequences for these immunoglobulins and their polypeptide domains. In certain embodiments, effective removal of a hinge region is determined based on a biological activity that is altered upon removal of the hinge. An example of biological activity that may be altered includes reduction of an isotype's ability to bind to some Fc receptors as for example, by modification or deletion of the hinge region as described herein.

As an example embodiments, the polypeptide comprising the C_{H}2 and C_{H}3 domains of a human IgG1 or a portion thereof may comprise SEQ ID NO: 8 or a sequence at least 90% identical thereto. The immunoglobulin peptide may be encoded by the nucleic acid sequence of SEQ ID NO: 9 or SEQ ID NO: 10.

For example, in one embodiment, the present invention comprises a nucleic acid encoding a non-immunoglobulin polypeptide directly linked to a polypeptide comprising a C_{H}2 domain of an immunoglobulin, or a fragment thereof (e.g., having the hinge sequences removed). In some embodiments, the C_{H}2 domain, or a fragment thereof, comprises SEQ ID NO: 14 or a sequence that is at least 70%, or 75%, or 80%, or 85%, or 90%, or 95%, or 96%, or 97%, or 98%, or 99% identical to SEQ ID NO: 14. In another embodiment, the fusion protein may comprise the C_{H}2 and C_{H}3 domains, or portions thereof, of a human IgG1. As an example embodiment, the polypeptide comprising the C_{H}2 and C_{H}3 domains of a human IgG1 may comprise SEQ ID NO: 8 or a sequence that is at least 70%, or 75%, or 80%, or 85%, or 90%, or 95%, or 96%, or 97%, or 98%, or 99% identical to SEQ ID NO: 8. The immunoglobulin peptide may be encoded by the nucleic acid sequence of SEQ ID NO: 9 or SEQ ID NO: 10, where silent base changes for the codons that encode for proline (CCG to CCC) and glycine (GGT to GGG) at the C-terminus of the sequence remove a cryptic RNA splice site near the terminal codon or a sequence that is at least 70%, or 75%, or 80%, or 85%, or 90%, or 95%, or 96%, or 97%, or 98%, or 99% identical to SEQ ID NO: 9 or SEQ ID NO: 10.

In one embodiment, the polypeptide may comprise an interdomain linker linked to a non-immunoglobulin polypeptide, where the interdomain linker replaces the hinge region of the immunoglobulin Fc polypeptide. In an embodiment, the non-immunoglobulin linker is N-terminal to the immunoglobulin, such that the C-terminal amino acid of the interdomain linker is directly linked to the N-terminal amino acid of a polypeptide comprising an immunoglobulin, or a fragment thereof. The non-immunoglobulin polypeptide may comprise a single or multiple domains. The immunoglobulin polypeptide may comprise a C_{H}2 domain of an immunoglobulin, or a portion thereof. In an embodiment, the immunoglobulin may comprise a polypeptide comprising the C_{H}2 and C_{H}3 domains of a human IgG1 or a portion of both, or either, of these domains.

An interdomain linker may be peptide sequence that is naturally downstream of, and thus, linked to, a particular domain. In one embodiment, where the first non-immunoglobulin domain is a member of the immunoglobulin supergene family but is not derived from an immunoglobulin or a fragment thereof, the first non-immunoglobulin domain may comprise the Receptor for Advanced Glycation Endproducts (RAGE) or a portion thereof such as the V domain. Or a RAGE variant or RAGE isoform polypeptide may be used. In alternate embodiments, the first interdomain linker may comprise an amino acid sequence as set forth in at least one of SEQ ID NOs: 3-7, or a sequence at least 90% identical thereto.

For example, for the RAGE V domain, the interdomain linker may comprise amino acid sequences that are naturally downstream from the V domain. In an embodiment, the linker may comprise SEQ ID NO: 3, corresponding to amino acids 117-123 of full-length RAGE. Or, the linker may comprise a peptide having additional portions of the natural sequence. For example, an interdomain linker comprising several amino acids (e.g., 1-3, 1-5, or 1-10, or 1-15 amino acids) upstream and downstream of SEQ ID NO: 3 may be used. Thus, in one embodiment, an interdomain linker for RAGE comprises SEQ ID NO: 5 comprising amino acids 117-136 of full-length RAGE. Or, fragments of SEQ ID NO: 5 deleting, for example, 1, 2, or 1-3, 1-5, 1-10, or 1-15 amino acids from either end of the linker may be used. In alternate embodiments, the linker may comprise a peptide that is at least 70% identical, or 80% identical, or 90% identical to SEQ ID NO: 3 or SEQ ID NO: 5.

In other embodiments, larger portions of the non-immunoglobulin polypeptide may be used. For example, in certain embodiments, the non-immunoglobulin polypeptide may comprise the RAGE V domain and the C1 domain (i.e., two domains) of RAGE. For the RAGE C1 domain, the interdomain linker may comprise peptide sequence that is naturally downstream of the C1 domain. In an embodiment, the interdomain linker may comprise SEQ ID NO: 4, corresponding to amino acids 222-251 of full-length RAGE. Or, the interdomain linker may comprise a peptide having additional portions of the natural RAGE sequence. For example, an interdomain linker comprising several (1-3, 1-5, or 1-10, or 1-15 amino acids) amino acids upstream and downstream of SEQ ID NO: 4 may be used. Or, fragments of SEQ ID NO: 4 may be used, deleting for example, 1-3, 1-5, or 1-10, or 1-15 amino acids from either end of the interdomain linker. For example, in one embodiment, a hinge-like domain may comprise SEQ ID NO: 6, corresponding to amino acids 222-226. In alternate embodiments, the linker may comprise a peptide that is at least 70% identical, or 80% identical, or 90% identical to SEQ ID NO: 4.

Or an RAGE interdomain linker may comprise SEQ ID NO: 7, corresponding to RAGE amino acids 318-342 downstream of the RAGE C2 domain may be used. Or, the linker may comprise several (1-3, 1-5, or 1-10, or 1-15 amino acids) amino acids upstream and downstream of SEQ ID NO: 7. Or, fragments of SEQ ID NO: 7 may be used, deleting for example, 1-3, 1-5, or 1-10, or 1-15 amino acids from either end of the interdomain linker. Or a peptide that is at least 70% identical, or 80% identical, or 90% identical to SEQ ID NO: 7 or a fragment thereof.

The method may also comprise the step of incorporating the DNA construct into an expression vector. The method may further comprise the step of transfecting a cell with the expression vector of the present invention. Thus, in an embodiment, the present invention comprises a cell transfected with the expression vector of the invention. For example, plasmids may be constructed to express fusion proteins by fusing different lengths of a 5′ cDNA sequence of non-immunoglobulin polypeptide with a 3′ cDNA sequence of a immunoglobulin polypeptide (e.g., IgG1 γ1). The expression cassette sequences may be inserted into an expression vector such as pcDNA3.1 expression vector (Invitrogen, CA) using standard recombinant techniques.

As is known in the art, such nucleic acid constructs may be modified by mutation, as for example, by PCR amplification of a nucleic acid template with primers comprising the mutation of interest. In this way, polypeptides comprising varying levels of biological activity may be designed. In alternate embodiments, the mutated sequences may be 70%, 75%, 80%, 85%, or 90% or more identical to the starting DNA. As such, variants may include nucleotide sequences that hybridize under stringent conditions (i.e., equivalent to about 20-27° C. below the melting temperature (TM) of the DNA duplex in 1 molar salt).

Also, the method may comprise transfecting the expression vector into a host cell. In certain embodiments, the fusion proteins of the present invention may be expressed in mammalian expression systems, including systems in which the expression constructs are introduced into the mammalian cells using virus such as retrovirus or adenovirus. Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines may be selected through determining which cell lines have high expression levels of the fusion protein. Other cell lines that may be used are insect cell lines, such as Sf9 cells. Plant host cells may include, e.g., Nicotiana, Arabidopsis, duckweed, corn, wheat, potato, and the like. Bacterial host cells may include E. coli and Streptomyces species. Yeast host cells include Schizosaccharomyces pombe, Saccharomyces cerevisiae and Pichia pastoris. When recombinant expression vectors encoding the fusion protein genes are introduced into mammalian host cells, the fusion proteins of the present invention may be produced by culturing the host cells for a period of time sufficient to allow for expression of the fusion protein in the host cells or secretion of the fusion protein into the culture medium in which the host cells are grown. The expressed fusion proteins may be recovered from the culture medium using standard protein purification methods.

Nucleic acid molecules encoding the fusion proteins of the present invention and expression vectors comprising these nucleic acid molecules may be used for transfection of a suitable mammalian, plant, bacterial or yeast host cell. Transformation may be performed by any known method for introducing polynucleotides into a host cell. Methods for introduction of heterologous polynucleotides into mammalian cells are known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming plant cells are known in the art, including, e.g., Agrobacterium-mediated transformation, biolistic transformation, direct injection, electroporation and viral transformation. Methods of transforming bacterial and yeast cells are also known in the art.

An expression vector may also be delivered to an expression system using DNA biolistics, wherein the plasmid is precipitated onto microscopic particles, preferably gold, and the particles are propelled into a target cell or expression system. DNA biolistics techniques are well-known the art and devices, e.g., a “gene gun”, are commercially available for delivery of the microparticles in to a cell (e.g., Helios Gene Gun, Bio-Rad Labs., Hercules, Calif.) and into the skin (PMED Device, PowderMed. Ltd., Oxford, UK).

Expression of the fusion proteins of the present invention from production cell lines may be enhanced using a number of known techniques. For example, the glutamine synthetase gene expression system (the GS system) and the plasma-encoded neomycin resistance system are common approaches for enhancing expression under certain conditions.

When the fusion proteins of the present invention are expressed by different cell lines, they may have different glycosylation patterns from each other. However, all fusion proteins encoded by the nucleic acid molecules as described herein, or comprising the amino acid sequences as described herein, are part of the instant invention, regardless of the glycosylation of the fusion protein.

In one embodiment, the recombinant DNA construct may be transfected into Chinese Hamster Ovary cells and expression optimized. In alternate embodiments, the cells may produce 0.1 to 20 grams/liter, or 0.5 to 10 grams/liter, or about 1-2 grams/liter.

For example, the recombinant vectors may be stably transfected into Chinese Hamster Ovary (CHO) cells, and cells expressing the RAGE fusion protein selected and cloned. In an embodiment, cells expressing the recombinant construct are selected for plasmid-encoded neomycin resistance by applying antibiotic G418. Individual clones may be selected and clones expressing high levels of recombinant protein as detected by Western Blot analysis of the cell supernatant may be expanded, and the gene product purified by affinity chromatography using Protein A columns.

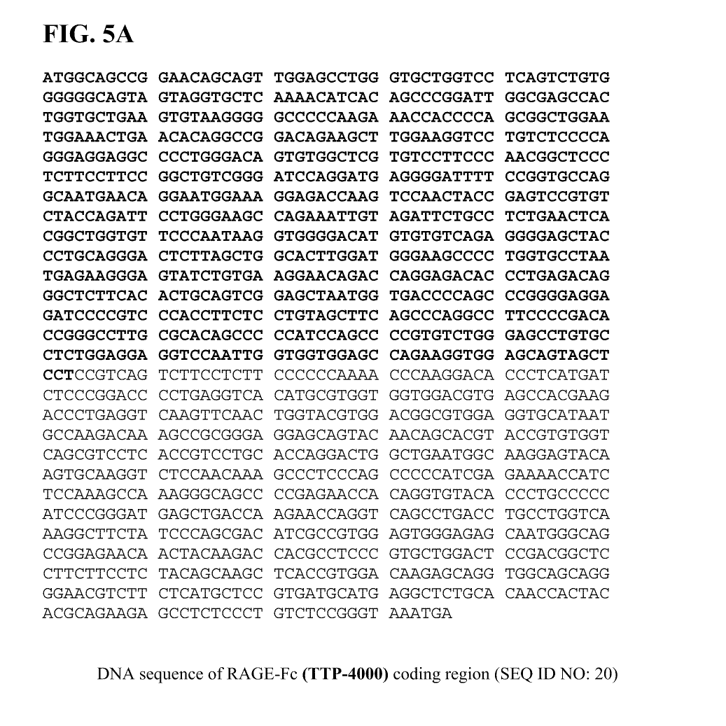

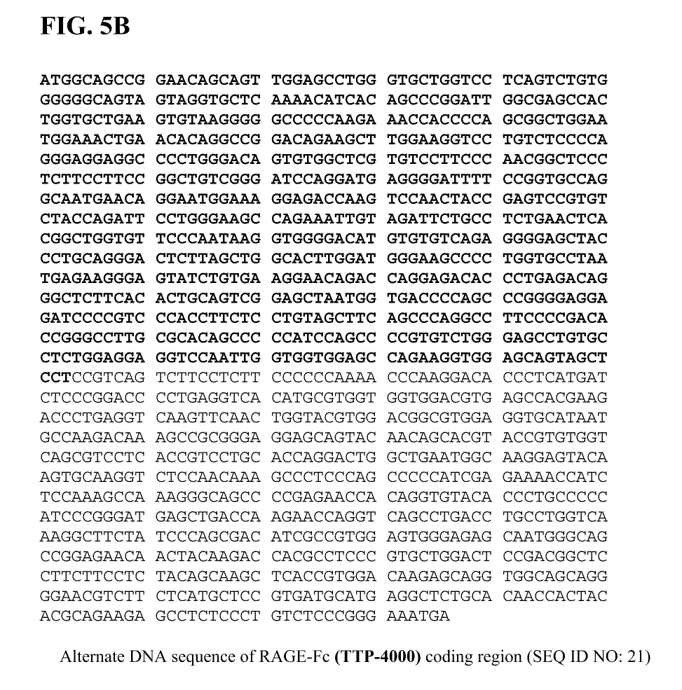

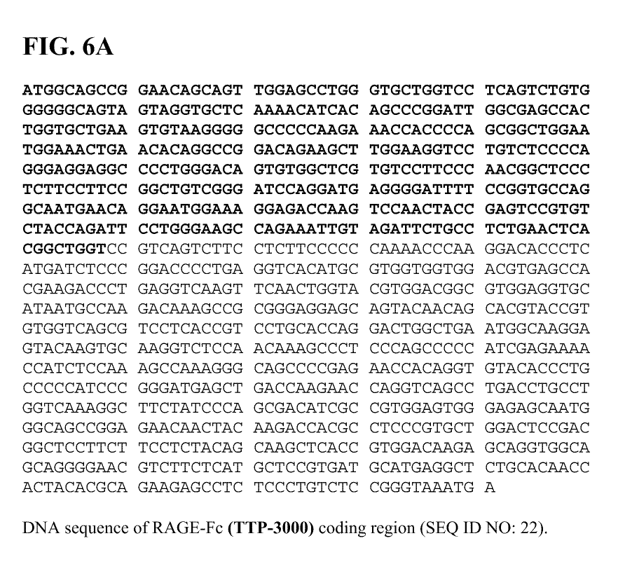

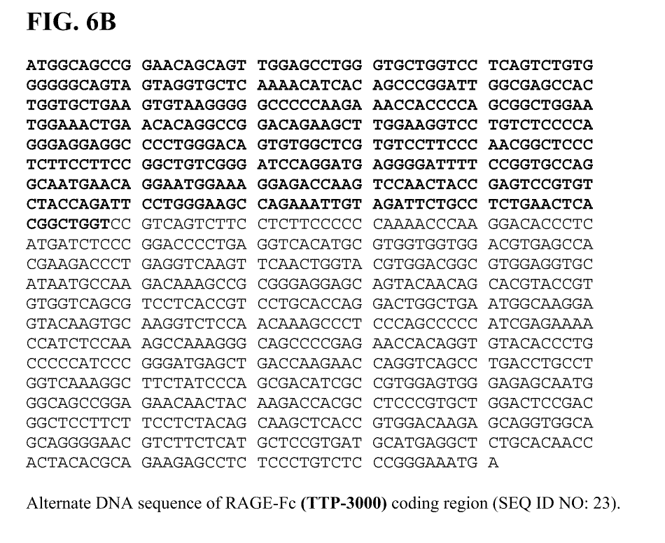

Sample embodiments of recombinant nucleic acids that encode fusion proteins of the present invention are shown in FIGS. 5 and 6. For example, as described above, the fusion protein produced by the recombinant DNA construct may comprise a non-immunoglobulin polypeptide that replaces an immunoglobulin Fc hinge region and that is operably linked to an immunoglobulin polypeptide. The fusion protein may comprise one or more domains derived from a non-immunoglobulin protein and one or more two domains derived from an immunoglobulin. An example nucleic acid construct encoding a RAGE fusion protein, TTP-4000 (TT4), having this type of structure is shown in FIG. 5A (SEQ ID NO: 20) and 5B (SEQ ID NO: 21). As shown in FIG. 5, coding sequence 1-753 (highlighted in bold) encodes the RAGE N-terminal protein sequence whereas the sequence from 754-1386 encodes the IgG Fc protein sequence. For the protein encoded by the nucleic acid sequence of FIG. 5, the first 251 amino acids of the full-length TTP-4000 RAGE fusion protein contains as the RAGE polypeptide sequence a signal sequence comprising amino acids 1-22/23, the RAGE V domain (including the ligand binding site) comprising amino acids 23/24-116, an interdomain linker comprising amino acids 117 to 123, a second RAGE domain (C1) comprising amino acids 124-221, and a downstream interdomain linker comprising amino acids 222-251.

In an embodiment, the fusion protein may not necessarily comprise a second non-immunoglobulin domain. For example, the fusion protein may comprise one non-immunoglobulin domain derived from RAGE and two immunoglobulin domains derived from a human Fc polypeptide. An example nucleic acid construct encoding this type of fusion protein is shown as FIG. 6A (SEQ ID NO: 22 and 6B SEQ ID NO: 23). As shown in FIG. 6, the coding sequence from nucleotides 1 to 408 (highlighted in bold) encodes the RAGE N-terminal protein sequence, whereas the sequence from 409-1041 codes the IgG1 (γ1) protein sequence. For the protein encoded by the nucleic acid sequence of FIG. 6, the first 136 amino acids of the full-length TTP-4000 RAGE fusion protein contains as the RAGE polypeptide sequence a signal sequence comprising amino acids 1-22/23, the RAGE V domain (including the ligand binding site) comprising amino acids 23/24-116, and an interdomain linker comprising amino acids 117 to 136.

The fusion proteins of the present invention may comprise improved in vivo stability and/or induce less inflammation as compared to fusion proteins comprising an immunoglobulin-based hinge region. The fusion protein may be further modified to increase stability, efficacy, potency and bioavailability. Thus, the fusion proteins of the present invention may be modified during translation or by post-translational processing or by chemical modification. For example, the fusion protein may be synthetically prepared to include L-, D-, or unnatural amino acids, alpha-disubstituted amino acids, or N-alkyl amino acids. Additionally, fusion proteins may be modified by acetylation, acylation, ADP-ribosylation, amidation, glycosylation, phosphorylation, derivatization by known protecting/blocking groups, proteolytic cleavage, attachment of lipids such as phosphatidyinositol, formation of disulfide bonds, and the like. Chemical modifications may include protection or modification of free —NH₂ groups, free —COOH groups, as well as modification of side groups of Trp, Tyr, Phe, His, Arg, Lys, as well as chemical cleavage (ie.g., cyanogen bromide, hydroxylamine, alkali and the like). Other modifications may include oxidation, reduction, and formylation. Furthermore, polyethylene glycol or other polymeric moieties as described herein can be added to increase the biological stability of the fusion protein.

### Binding of Ligands to Fusion Proteins of the Invention

The fusion proteins of the present invention may comprise a number of applications. As noted herein, in some embodiments, the non-immunoglobulin polypeptide domain may comprise a ligand binding domain or ligand binding site, or a portion of a ligand binding domain or ligand binding site. Also, in certain embodiments, the immunoglobulin polypeptide domains may comprise an uninterrupted portion of a ligand binding domain or an uninterrupted portion of ligand binding site. In such embodiments, the fusion protein of the present invention may be used in a binding assay to identify ligands that bind to the non-immunoglobulin polypeptide, such as agonists, antagonists, or modulators.

For example, in one embodiment, the present invention provides a method for detection of ligands or modulators comprising: (a) providing a fusion protein comprising a non-immunoglobulin polypeptide linked to an immunoglobulin polypeptide, where the non-immunoglobulin polypeptide comprises a ligand binding site and the immunoglobulin polypeptide does not include a functional hinge domain; (b) mixing a compound of interest and a ligand having a known binding affinity for the non-immunoglobulin polypeptide with the fusion protein; and (c) measuring binding of the known ligand to the fusion protein in the presence of the compound of interest.

The fusion proteins may also provide kits for the detection of ligands and/or modulators that bind to the non-immunoglobulin polypeptide. For example, in one embodiment, a kit of the present invention may comprise: (a) a compound having known binding affinity to the non-immunoglobulin polypeptide as a positive control; (b) a fusion protein comprising a non-immunoglobulin polypeptide linked to an immunoglobulin polypeptide, wherein the non-immunoglobulin polypeptide comprises a ligand and/or modulator binding site and the immunoglobulin polypeptide does not include a functional hinge domain; and (c) instructions for use. In embodiments of the assays and kits of the invention, the fusion protein may comprise fusion proteins as described herein comprising: (a) an immunoglobulin polypeptide from which the hinge region has been removed, and (b) a non-immunoglobulin polypeptide comprising an uninterrupted portion of an immunoglobulin supergene protein. In some embodiments, wherein the uninterrupted portion of the immunoglobulin supergene protein may comprise a first non-immunoglobulin polypeptide domain directly linked to the N-terminal end of a first interdomain linker. In one embodiment, and the immunoglobulin polypeptide may be operably linked to the C-terminal end of the first interdomain linker. In an embodiment, the immunoglobulin polypeptide is directly linked to the C-terminal end of the first interdomain linker. Or, other embodiments of the fusion proteins of the present invention may be used.

For example, the fusion protein may be used in a binding assay to identify potential ligands that bind to the non-immunoglobulin protein. In one example embodiment of such a binding assay, a known ligand may coated onto a solid substrate (e.g., Maxisorb plates) at a biologically relevant concentration (e.g., about 5 micrograms per well), where each well contains a total volume of about 100 microliters (μL). The plates may be incubated at 4° C. overnight to allow the ligand to bind to the substrate. Alternatively, shorter incubation periods at higher temperature (e.g., room temperature) may be used. After a period of time to allow for the ligand to bind to the substrate, the assay wells may be aspirated and a blocking buffer (e.g., 1% BSA in 50 mM imidizole buffer, pH 7.2) may be added to block nonspecific binding. For example, blocking buffer may be added to the plates for 1 hour at room temperature. The plates may then be aspirated and/or washed with a wash buffer. In one embodiment, a buffer comprising 20 mM Imidizole, 150 mM NaCl, 0.05% Tween-20, 5 mM CaCl₂ and 5 mM MgCl₂, pH 7.2, may be used as a wash buffer. Or, other wash buffers used for ELISA type binding assays may be used. The fusion protein may then be added at increasing dilutions to the assay wells. The fusion protein may then be allowed to incubate with the immobilized ligand in the assay well such that binding can attain equilibrium. In one embodiment, the fusion protein is allowed to incubate with the immobilized ligand for about one hour at 37° C. In alternate embodiments, longer incubation periods at lower temperatures may be used. After the fusion protein and immobilized ligand have been incubated, the plate may be washed to remove any unbound fusion protein. The fusion protein bound to the immobilized ligand may be detected in a variety of ways. In one embodiment, detection employs an ELISA. Where the fusion protein comprises an IgG1 polypeptide as the immunoglobulin, an immunodetection complex containing a monoclonal mouse anti-human IgG1, biotinylated goat anti-mouse IgG, and an avidin linked alkaline phosphatase may be added to the fusion protein immobilized in the assay well. The immunodetection complex may be allowed to bind to the immobilized fusion protein such that binding between the fusion protein and the immunodetection complex attains equilibrium. For example, the complex may be allowed to bind to the fusion protein for about one hour at room temperature (or for longer times at lower temperatures). At that point, any unbound complex may be removed by washing the assay well with wash buffer. The bound complex may be detected by adding a substrate for the secondary antibody complex, e.g., the alkaline phosphatase substrate, para-nitrophenylphosphate (PNPP), and measuring conversion of PNPP to para-nitrophenol (PNP) as an increase in absorbance at 405 nm.

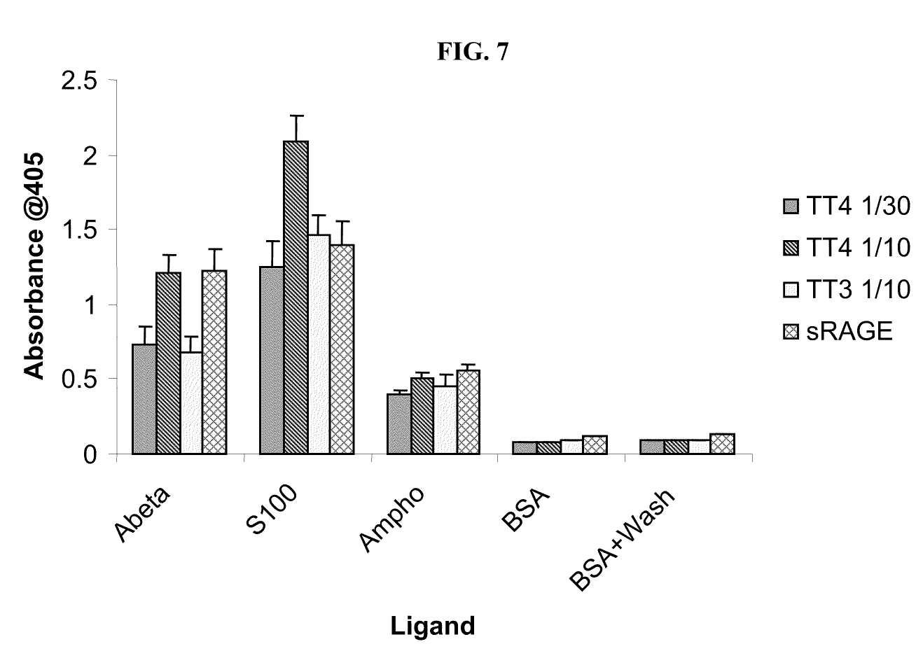

In an embodiment, a ligand may bind to the fusion protein with nanomolar (nM) or micromolar (μM) affinity. An experiment illustrating binding of RAGE ligands to a RAGE fusion protein of the present invention is shown in FIG. 7. Solutions of TTP-3000 (TT3) and TTP-4000 (TT4) having initial concentrations of 1.082 mg/mL, and 370 μg/mL, respectively, were prepared. As shown FIG. 7, at various dilutions, the RAGE fusion protein TTP-4000 was able to bind to immobilized RAGE ligands Amyloid-beta (Abeta) (Amyloid Beta (1-40) from Biosource), S100b (S100), and amphoterin (Ampho), resulting in an increase in absorbance. In the absence of ligand (i.e., coating with only BSA) there was no increase in absorbance. The binding assay of the present invention may be used to quantify ligand binding. For example, in alternate embodiments, RAGE ligands may bind to the RAGE fusion protein TTP-4000 with binding affinities ranging from 0.1 to 1000 nanomolar (nM), or from 1 to 500 nM, or from 50 to 200 nM, or from 10 to 80 nM (e.g., as measured by binding dissociation constant or K_{D}).

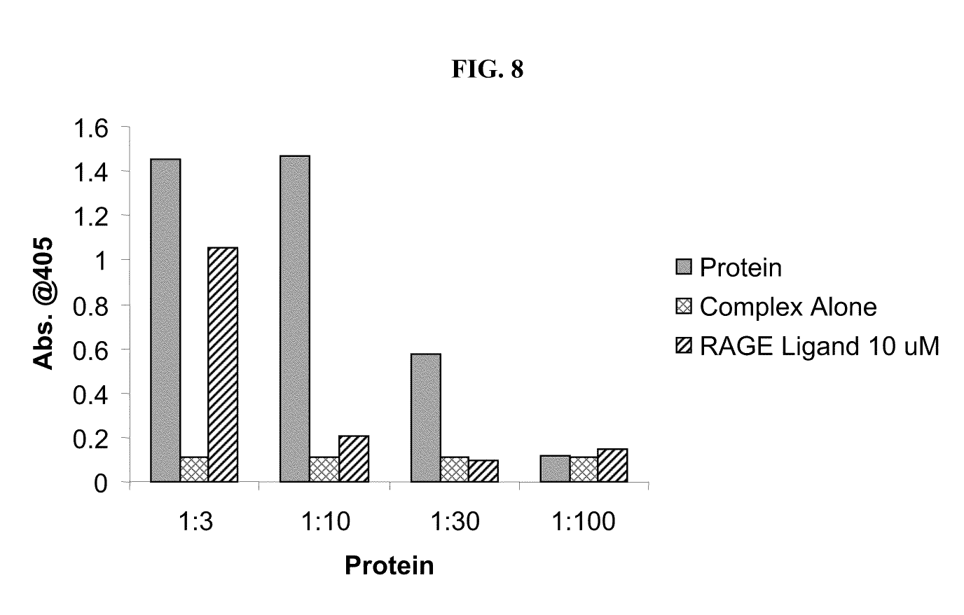

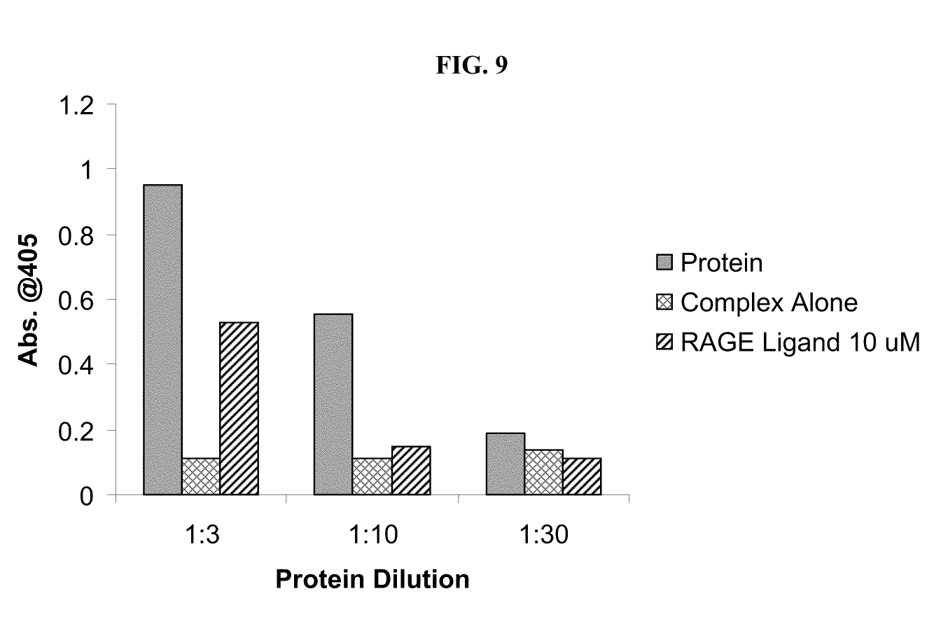

The fusion protein of the present invention may also be used to identify compounds having the ability to bind to the non-immunoglobulin polypeptide. As shown in FIGS. 8 and 9, respectively, a RAGE ligand may be assayed for its ability to compete with immobilized amyloid beta for binding to TTP-4000 (TT4) or TTP-3000 (TT3) RAGE fusion proteins. Thus, it may be seen that a RAGE ligand at a final assay concentration (FAC) of 10 μM can displace binding of RAGE fusion protein to amyloid-beta at concentrations of 1:3, 1:10, 1:30, and 1:100 of the initial TTP-4000 solution (i.e., 370 μg/mL) (FIG. 8) or initial TTP-3000 solution (1 mg/mL) (FIG. 9).

### Modulation of Cellular Effectors

Embodiments of the fusion proteins of the present invention may be used to modulate a biological response. In an embodiment, fusion proteins of the present invention may be used to modulate the function of an enzyme. For example, fusion proteins having RAGE or a fragment thereof as the non-immunoglobulin protein may be designed to modulate RAGE-induced increases in gene expression. For example, the interaction between RAGE and its ligands may generate oxidative stress and activation of NF-κB, and NF-κB regulated genes, such as the cytokines IL-1β, TNF-α, and the like.

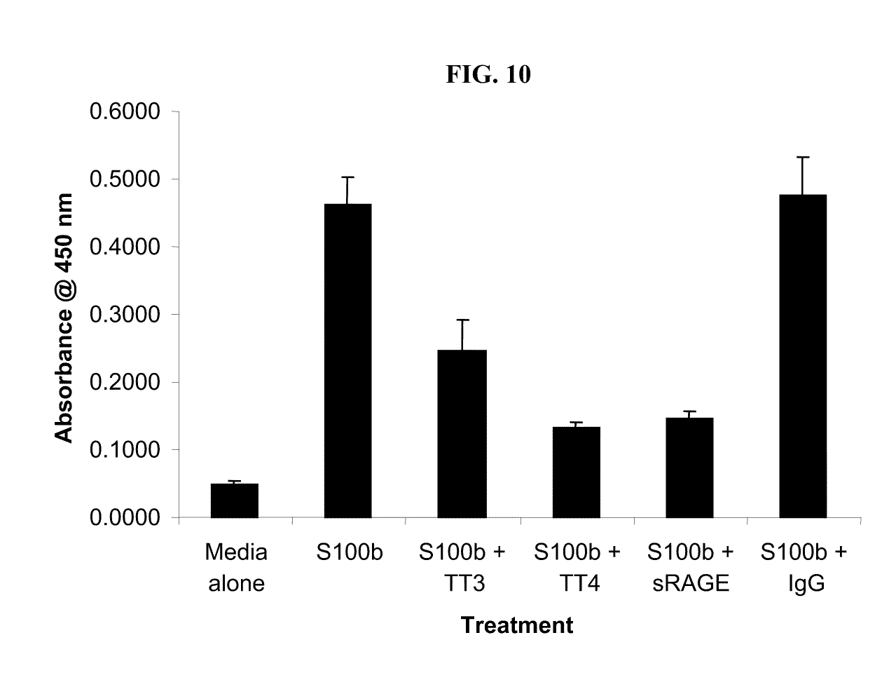

One embodiment of a use of a RAGE fusion protein of the present invention to modulate expression of the cellular effector TNF-α is shown in FIG. 10. THP-1 myeloid cells may be cultured in RPMI-1640 media supplemented with 10% FBS and induced to secrete TNF-α via stimulation of RAGE with S100b. When such stimulation occurs in the presence of a RAGE fusion protein of the invention, induction of TNF-α by S100b binding to RAGE may be inhibited. Thus, as shown in FIG. 10, addition of 10 μg TTP-3000 (TT3) or TTP-4000 (TT4) RAGE fusion protein reduces S100b induction of TNF-α by about 50% to 75%, respectively. RAGE fusion protein TTP-4000 may be at least as effective in blocking S100b induction of TNF-α as is sRAGE (FIG. 10). Specificity of the inhibition for the RAGE sequences of TTP-4000 and TTP-3000 is shown by the experiment in which IgG alone was added to S100b stimulated cells. Addition of IgG and S100b to the assay shows the same levels of TNF-α as S100b alone.

### Physiological Characteristics of Fusion Proteins Lacking a Hinge Domain

Linking a non-immunoglobulin polypeptide to an immunoglobulin polypeptide may increase the half-life of the non-immunoglobulin polypeptide in serum. Additionally, removing the hinge region from the immunoglobulin portion of the fusion protein, may further increase the half-life of the construct, by reducing the inflammatory response generated by a subject exposed to the fusion protein.

For example, while sRAGE can have a therapeutic benefit in the modulation of RAGE-mediated diseases (by inhibiting the interaction of RAGE ligands with RAGE in the cell membrane), human sRAGE may have limitations as a stand-alone therapeutic based on the relatively short half-life of sRAGE in plasma. Thus, whereas rodent sRAGE has a half-life in normal and diabetic rats of approximately 20 hours, human sRAGE has a half-life of less than 2 hours when assessed by retention of immunoreactivity sRAGE (Renard et al., J. Pharmacol. Exp. Ther., 290:1458-1466 (1999)). As therapeutics for RAGE-mediated diseases, however, RAGE fusion proteins may not require the generation of an inflammatory response. To generate a RAGE therapeutic that has similar binding characteristics as sRAGE, but a more stable pharmacokinetic profile, a RAGE fusion protein comprising a RAGE ligand binding site linked to one or more human immunoglobulin domains may be used. In an embodiment, the immunoglobulin domains may include the Fc portion of the immunoglobulin heavy chain, but may have the hinge region removed.

Thus, embodiments of the fusion proteins of the present invention may comprise a fusion protein comprising a polypeptide linked to an immunoglobulin domain(s) where the Fc hinge region from the immunoglobulin is removed and replaced with a non-immunoglobulin polypeptide. In this way, interaction between the fusion protein and Fc receptors on inflammatory cells may be minimized. It may be important, however, to maintain proper stacking and other three-dimensional structural interactions between the various immunoglobulin domains of the fusion protein. Thus, in certain embodiments, the first non-immunoglobulin domain and the first interdomain linker may be derived from a protein that is a member of the immunoglobulin supergene family. Also, in certain embodiments, the first interdomain linker is directly linked to the first non-immunoglobulin domain in the protein from which the first non-immunoglobulin polypeptide and first interdomain linker are both derived. For example, the interdomain linker may be a portion of the non-immunoglobulin protein that is naturally downstream or naturally upstream of the first non-immunoglobulin polypeptide domain. Thus, in certain embodiments, the fusion proteins comprise a polypeptide domain derived from a non-immunoglobulin protein having the portion of the protein that is naturally downstream or upstream of the domain still attached to the domain. In an embodiment, the fusion protein may comprise: (a) an immunoglobulin polypeptide from which the hinge region has been removed, and (b) a non-immunoglobulin polypeptide comprising an uninterrupted portion of an immunoglobulin supergene protein. In certain embodiments, the uninterrupted portion of the immunoglobulin supergene protein may comprise a first non-immunoglobulin polypeptide domain directly linked to the N-terminal end of a first interdomain linker. Also, in some embodiments, the immunoglobulin polypeptide may be operably linked to the C-terminal end of the first interdomain linker. In an embodiment, the immunoglobulin polypeptide is directly linked to the C-terminal end of the first interdomain linker.

In some embodiments, the non-immunoglobulin polypeptide domain may comprise a ligand binding domain or ligand binding site, or a portion of a ligand binding domain or ligand binding site. Also, in certain embodiments, the immunoglobulin polypeptide domains may comprise an uninterrupted portion of a ligand binding domain or an uninterrupted portion of ligand binding site.

In one embodiment, the non-immunoglobulin polypeptide is a member of the immunoglobulin supergene family but is not derived from an immunoglobulin or a fragment thereof. Thus, members of the immunoglobulin supergene family that do not function as prototypical immunoglobulins may be used as the source of the non-immunoglobulin polypeptide. Or, isoforms or biologically active variants of such proteins may be used. For example, such proteins may include RAGE, T cell receptors, Killer-cell immunoglobulin-like receptors (KIR), proteins that comprise the class I or class II major histocompatibility complex (MHC class I), CD2, the CD3 receptor (γ, δ and ε chains), CD4, CD8, CD28, CD79a and CD79b, CD80 and CD86 (i.e., B7.1 and B7.2), CD147, LFA, and CTLA4, certain Fc receptors, Intercellular adhesion molecules (ICAMs), Vascular cell adhesion molecule-1 (VCAM-1), Neural Cell Adhesion Molecule (NCAM), Interleukin-1 receptor type I, Platelet-derived growth factor receptor (PDGFR), and Thymocyte differentiation antigen-1 (i.e., Thy-1 or CD90), or biologically active variants and/or isoforms of such proteins. In alternate embodiments, the first non-immunoglobulin polypeptide domain and the first interdomain linker comprise an uninterrupted portion of a polypeptide derived from at least one of RAGE, LFA-3, CTLA, or CD4. In an embodiment, the first non-immunoglobulin protein may comprise the Receptor for Advanced Glycation Endproducts (RAGE) or a portion thereof.

Thus, embodiments of the fusion proteins of the present invention may substitute the biologically inert, but structurally similar RAGE interdomain linker that separates the V and C1 domains of RAGE, or the linker that separates the C1 and C2 domains of RAGE, in lieu of the normal hinge region of the immunoglobulin heavy chain. The RAGE polypeptide of the RAGE fusion protein may comprise an interdomain linker sequence that is naturally found downstream of a RAGE domain to form a RAGE domain/linker fragment. In this way, the three dimensional interactions between the domains contributed by either RAGE or the immunoglobulin may be maintained. Or, other embodiments of the fusion proteins of the present invention may be used.

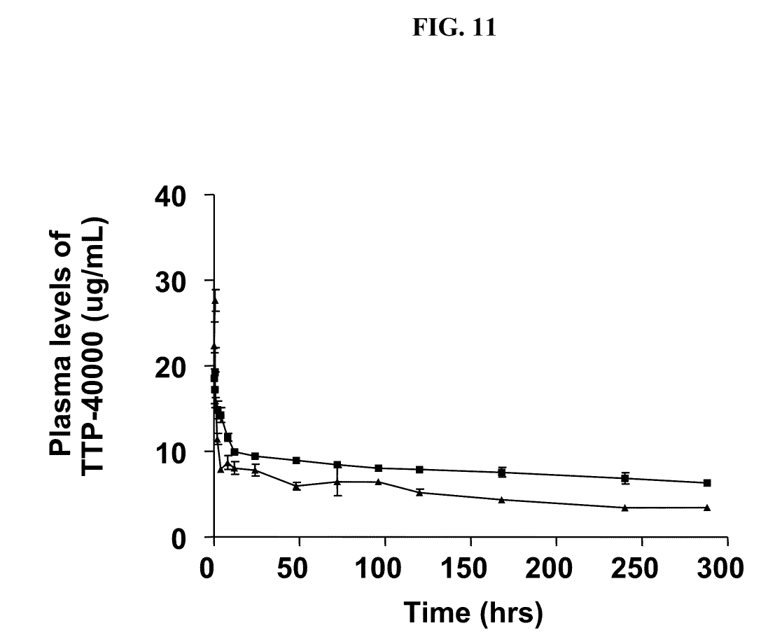

In an embodiment, a RAGE fusion protein of the present invention may comprise a substantial increase in pharmacokinetic stability as compared to sRAGE. For example, FIG. 11 shows that once the RAGE fusion protein TTP-4000 has saturated its ligands, it may retain a half-life of greater than 300 hours. This may be contrasted with the half-life for sRAGE of only a few hours in human plasma.

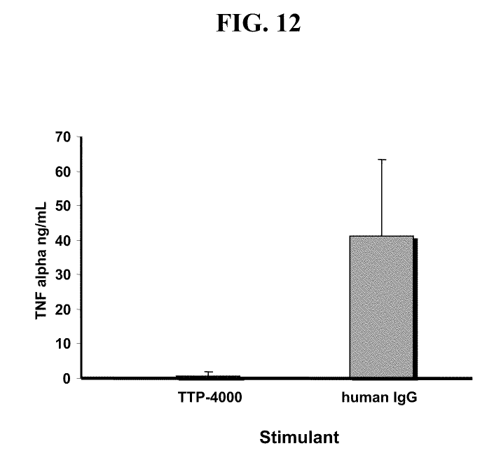

Thus, in an embodiment, the RAGE fusion proteins of the present invention may be used to antagonize binding of physiological ligands to RAGE as a means to treat RAGE-mediated diseases without generating an unacceptable amount of inflammation. The fusion proteins of the present invention may exhibit a substantial decrease in generating a proinflammatory response as compared to IgG. For example, as shown in FIG. 12, the RAGE fusion protein TTP-4000 does not stimulate TNF-α release from cells under conditions where human IgG stimulation of TNF-α release is detected.

Treatment of Disease with Fusion Proteins

The present invention may also comprise using the fusion proteins of the invention for the treatment of disorder in a human subject. In an embodiment, the method may comprise administering to a subject a fusion protein comprising a non-immunoglobulin polypeptide linked to an immunoglobulin polypeptide. In certain embodiments, the present invention comprises a method of treating a disorder in a subject comprising administering to a subject a composition comprising a therapeutically effective amount of a fusion protein comprising: (a) an immunoglobulin polypeptide from which the hinge region has been removed; and (b) a non-immunoglobulin polypeptide comprising an uninterrupted portion of an immunoglobulin supergene protein, wherein the uninterrupted portion of the immunoglobulin supergene protein comprises a first non-immunoglobulin polypeptide domain directly linked to the N-terminal end of a first interdomain linker, and wherein the immunoglobulin polypeptide is operably linked to the C-terminal end of the first interdomain linker. In one embodiment, the immunoglobulin polypeptide is directly linked to the C-terminal end of the first interdomain linker. In an embodiment, the disorder is mediated at least in part by the non-immunoglobulin polypeptide.

As the hinge region of the immunoglobulin chain may be proinflammatory in vivo, embodiments of the fusion proteins of the present invention substitute an amino acid sequence derived from a non-immunoglobulin derived protein for the hinge domain of the immunoglobulin. In certain embodiments, the non-immunoglobulin polypeptide may comprise a region, as for example, an interdomain linker, which replaces the immunoglobulin hinge region in a manner that allows for correct assembly of the immunoglobulin polypeptide.

In an embodiment, the interdomain linker sequence from the non-immunoglobulin polypeptide does not promote binding of the fusion protein to a Fc receptor (FcR) at physiological or therapeutically effective concentrations of the fusion protein. For example, in alternate embodiments, replacement of an immunoglobulin hinge region with an interdomain linker from the non-immunoglobulin protein may reduce binding of the fusion protein to the Fc receptor by at least 2-fold, or 5-fold, or 10-fold, or 20-fold, or 50-fold, or 100-fold, or 500-fold, or 1.000-fold, or 10,000 fold or greater as compared to an immunoglobulin polypeptide having the hinge region.

Also in some embodiments, the fusion proteins of the present invention may induce less inflammation when administered to a subject as compared to fusion proteins having functional Fc hinge sequences. For example, in alternate embodiments, replacement of an immunoglobulin hinge region with an interdomain linker from the non-immunoglobulin protein may reduce the ability of the fusion protein to induce inflammation by as compared to an immunoglobulin polypeptide having the hinge region.

For example, in certain embodiments, the fusion protein may comprise an immunoglobulin polypeptide from which the hinge region has been removed, and a non-immunoglobulin polypeptide. In an embodiment, the non-immunoglobulin polypeptide may comprise a first non-immunoglobulin domain. Also, the non-immunoglobulin polypeptide may comprise a first interdomain linker.

In certain embodiments, the first non-immunoglobulin domain and the first interdomain linker may be derived from a protein that is a member of the immunoglobulin supergene family. Also, in certain embodiments, the first interdomain linker is directly linked to the first non-immunoglobulin domain in the protein from which the first non-immunoglobulin polypeptide and first interdomain linker are both derived.

The interdomain linker may be a portion of the non-immunoglobulin protein that is naturally downstream or naturally upstream of the first non-immunoglobulin polypeptide domain. In alternate embodiments, linkers as described herein may be used. The non-immunoglobulin polypeptide and its associated linker may be upstream (N-terminal) or downstream (C-terminal) of the immunoglobulin polypeptide. In an embodiment, the first interdomain linker is positioned between the first non-immunoglobulin domain and the immunoglobulin portion of the fusion protein. For example, in an embodiment, the N-terminal amino acid of the first interdomain linker is linked to the C-terminal amino acid of the first non-immunoglobulin domain and the C-terminal amino acid of the first interdomain linker is linked to the N-terminal amino acid of an immunoglobulin domain. For example, the C-terminal amino acid of the first interdomain linker may be directly linked to the N-terminal amino acid of a polypeptide comprising a C_{H}2 domain of an immunoglobulin, or a portion thereof. In an embodiment, the C_{H}2 domain has the hinge region removed. In an embodiment, the first interdomain linker replaces the Fc hinge region of the immunoglobulin. In certain embodiments, the first interdomain linker is positioned to replace the immunoglobulin Fc hinge region to allow for correct assembly of the immunoglobulin polypeptide domain. Also in certain embodiments, the first interdomain linker linked to the first non-immunoglobulin domain does not promote binding of the fusion protein to a Fc receptor (FcR) at a therapeutically effective concentration of the fusion protein.

For example, in certain embodiments of the methods of treatment of the invention, the fusion protein may comprise: (a) an immunoglobulin polypeptide from which the hinge region has been removed, and (b) a non-immunoglobulin polypeptide comprising an uninterrupted portion of an immunoglobulin supergene protein. In certain embodiments, the uninterrupted portion of the immunoglobulin supergene protein comprises a first non-immunoglobulin polypeptide domain directly linked to the N-terminal end of a first interdomain linker. Also in some embodiments, the immunoglobulin polypeptide is operably linked to the C-terminal end of the first interdomain linker. In an embodiment, the uninterrupted portion of the immunoglobulin supergene protein comprises a first non-immunoglobulin polypeptide domain directly linked to the N-terminal end of a first interdomain linker, wherein the immunoglobulin polypeptide is operably linked to the C-terminal end of the first interdomain linker. In one embodiment, the immunoglobulin polypeptide is directly linked to the C-terminal end of the first interdomain linker.

The immunoglobulin polypeptide and the non-immunoglobulin polypeptide may be from the same species, or from different species. In an embodiment, the immunoglobulin polypeptide and the non-immunoglobulin polypeptide are from the same species. For example, in an embodiment, both the immunoglobulin polypeptide and the human non-immunoglobulin polypeptide are derived from human proteins.

In an embodiment, the immunoglobulin polypeptide comprises at least one immunoglobulin constant domain or a portion thereof. For example, the immunoglobulin polypeptide may comprise at least one of a C_{H}2 domain or a C_{H}3 domain of an immunoglobulin, or a portion of the C_{H}2 domain or the C_{H}3 domain of an immunoglobulin. In certain embodiments, the immunoglobulin polypeptide may comprise an Fc fragment without the hinge region. For example, in some embodiments, the immunoglobulin polypeptide may comprise a portion of the C_{H}2 domain and at least a portion of the C_{H}3 domain of an immunoglobulin. The portion of the C_{H}2 domain may comprise the C_{H}2 domain with the hinge region removed. In one embodiment, the immunoglobulin polypeptide comprises a portion of the C_{H}2 domain and the C_{H}3 domain of an immunoglobulin. In one embodiment, the C_{H}2 domain has the Fc hinge region removed. For example, in one embodiment, the C_{H}2 domain comprises SEQ ID NO: 8, or a portion of SEQ ID NO: 8, or a sequence at least 70%, 75%, 80%, 85%, 90%, 95%, 96%, 97%, 98%, or 99% identical thereto.

In other embodiments, the non-immunoglobulin polypeptide may comprise a plurality of domains. In some embodiments, the multiple domains may be linked to each other by an interdomain linker(s). Thus, in certain embodiments, the fusion protein of the present invention may comprise multiple non-immunoglobulin polypeptide domains linked to each other by one or more interdomain linkers. For example, in some embodiments, the non-immunoglobulin polypeptide further comprises at least one additional non-immunoglobulin polypeptide domain operably linked to the first non-immunoglobulin domain. The additional domain(s) may be positioned upstream or downstream to the first non-immunoglobulin domain. In an embodiment, the non-immunoglobulin polypeptide further comprises a second non-immunoglobulin polypeptide domain that is operably linked to the N-terminal end of the first non-immunoglobulin polypeptide domain. In certain embodiments, the second non-immunoglobulin polypeptide domain is directly linked to the first non-immunoglobulin polypeptide domain. In other embodiments, there may be a second interdomain linker between the first and second non-immunoglobulin domains. In some embodiments, the first and second interdomain linkers have different amino acid sequences. In an embodiment, the additional domain(s) may be positioned distal to the immunoglobulin portion of the fusion protein as compared to the first immunoglobulin domain. Thus, if the non-immunoglobulin domain(s) are upstream of the immunoglobulin portion of the fusion protein, the additional domains may be positioned upstream of the first non-immunoglobulin domain and first interdomain linker. Alternatively, if the non-immunoglobulin domain(s) are downstream of the immunoglobulin portion of the fusion protein, the additional domains may be positioned downstream of the first non-immunoglobulin domain and first interdomain linker. In yet other embodiments, the fusion protein may comprise non-immunoglobulin domains that are positioned both N-terminal and C-terminal to an immunoglobulin domain, or immunoglobulin domains that are positioned both N-terminal and C-terminal to a non-immunoglobulin domain.

In some embodiments, the non-immunoglobulin polypeptide domain may comprise a ligand binding domain or ligand binding site, or a portion of a ligand binding domain or ligand binding site. Also, in certain embodiments, the immunoglobulin polypeptide domains may comprise an uninterrupted portion of a ligand binding domain or an uninterrupted portion of ligand binding site.

In one embodiment, first non-immunoglobulin polypeptide is a member of the immunoglobulin supergene family but is not derived from an immunoglobulin or a fragment thereof. Thus, members of the immunoglobulin supergene family that do not function as prototypical immunoglobulins may be used as the source of the non-immunoglobulin polypeptide. Such proteins include the following proteins, or biologically active variants or isoforms of such proteins: RAGE, T cell receptors, Killer-cell immunoglobulin-like receptors (KIR), proteins that comprise the class I or class II major histocompatibility complex (MHC class I), CD2, the CD3 receptor (γ, δ and ε chains), CD4, CD8, CD28, CD79a and CD79b, CD80 and CD86 (i.e., B7.1 and B7.2), CD147, LFA, and CTLA4, certain Fc receptors, Intercellular adhesion molecules (ICAMs), Vascular cell adhesion molecule-1 (VCAM-1), Neural Cell Adhesion Molecule (NCAM) Interleukin-1 receptor type I, Platelet-derived growth factor receptor (PDGFR), and Thymocyte differentiation antigen-1 (i.e., Thy-1 or CD90). In alternate embodiments, the first non-immunoglobulin polypeptide domain and the first interdomain linker comprise an uninterrupted portion of a polypeptide derived from at least one of RAGE, LFA-3, CTLA, or CD4. Or, other embodiments of the fusion proteins of the present invention as described herein may be used.

In an embodiment, a fusion protein of the present invention may be administered by various routes. Administration of the fusion protein of the present invention may employ a parenteral route. For example, administration of the fusion protein of the present invention may employ intraperitoneal (IP) injection. In another embodiment, administration is intravenous (IV). The fusion protein may also be injected subcutaneously. In another embodiment, administration of the fusion protein is intra-arterial. Alternatively, the fusion protein may be administered orally, intranasally, or as an aerosol. In another embodiment, administration is sublingual. Also, administration may employ a time-release capsule. In yet another embodiment, administration may be transrectal, as by a suppository or the like. For example, subcutaneous administration may be useful to treat chronic disorders when the self-administration is desirable.

For example, fusion proteins of the present invention comprising a RAGE ligand binding site have been used in the treatment of diabetes related pathology. The RAGE fusion protein TTP-4000 was evaluated in a diabetic rat model of restenosis which involved measuring smooth muscle proliferation and intimal expansion following vascular injury. Use of the RAGE fusion proteins in the treatment of diabetes related pathology is illustrated in Table 1. The RAGE fusion protein TTP-4000 was evaluated in a diabetic rat model of restenosis which involved measuring smooth muscle proliferation and intimal expansion following vascular injury. As illustrated in Table 1, TTP-4000 treatment may significantly reduce the intima/media (I/M) ratio in diabetes-associated restenosis in a dose-responsive manner. Also, TTP-4000 treatment may significantly reduce restenosis-associated vascular smooth muscle cell proliferation in a dose-responsive manner.

TABLE 1
Effect of TTP-4000 in Rat Model of Restenosis

| | | TTP-4000 (n = 9) | TTP-4000 (n = 9) |
|---|---|---|---|
| | | Low dose** (0.3 mg/ | High dose** (1.0 mg/ |
| | IgG (n = 9) | animal qod × 4) | animal qod × 4) |
| Intimal area (mm²) | 0.2 ± 0.03 | 0.18 ± 0.04 | 0.16 ± 0.02 |
| Medial area (mm²) | 0.12 ± 0.01 | 0.11 ± 0.02 | 0.11 ± 0.01 |
| I/M ratio | 1.71 ± 0.27 | 1.61 ± 0.26 | 1.44* ± 0.15 |

*P < 0.05;
**For both high and low dose, a loading dose of 3 mg/animal was used

Also, fusion proteins of the present invention comprising a RAGE ligand binding site may also be used to treat reduce amyloidosis and to reduce amyloid plaques and cognitive dysfunction associated with Alzheimer's Disease (AD). It has been found that mice that have AD, and are treated for 3 months with either TTP-4000 had fewer amyloid beta (Aβ) plaques and less cognitive dysfunction than animals that received a vehicle or a human IgG negative control (IgG1).

Also, fusion proteins of the present invention comprising a RAGE ligand binding site may be used to treat atherosclerosis and other cardiovascular disorders such as stroke. When TTP-4000 was compared to sRAGE in a disease relevant animal model of stroke, TTP-4000 was found to provide a significantly greater reduction in infarct volume (Table 2). In this model, the middle carotid artery of a mouse is ligated and then reperfused to form an infarct. To assess the efficacy of RAGE fusion proteins to treat or prevent stroke, mice were treated with sRAGE or TTP-4000 or control immunoglobulin just prior to reperfusion. It can be seen that TTP-4000 was more efficacious than sRAGE in limiting the area of infarct in these animals suggesting that TTP-4000, because of its better half-life in plasma, was able to maintain greater protection than sRAGE.

TABLE 2
Reduction of Infarct in Stroke

| | % Reduction of |
|---|---|
| | Infarct** |
| sRAGE | 15%* |
| TTP-4000 (300 μg) | 38%* |
| TTP-4000 (300 μg) | 21%* |
| TTP-4000 (300 μg) | 10%* |
| IgG Isotype control | 4% |
| (300 μg) | |

*Significant to p < 0.001;
**Compared to saline

In another embodiment, a RAGE fusion proteins of the present invention may be used to treat cancer. In yet another embodiment, the RAGE fusion proteins of the present invention may be used to treat inflammation. In alternate embodiments, the RAGE fusion proteins of the present invention may be used to treat inflammation associated with inflammatory bowel disease, inflammation associated with rheumatoid arthritis, inflammation associated with psoriasis, inflammation associated with multiple sclerosis, inflammation associated with hypoxia, inflammation associated with stroke, inflammation associated with heart attack, inflammation associated with hemorrhagic shock, inflammation associated with sepsis, inflammation associated with organ transplantation, inflammation associated with impaired wound healing, or inflammation associated with rejection of self (e.g., autoimmune) or non-self (e.g., transplanted) cells, tissue, or organs. In an embodiment, the RAGE fusion proteins of the present invention may be used to treat auto-immune based disorders such as type I diabetes. Accordingly, the sRAGE fusion proteins of the present invention may be used to: (1) reduce onset of type I diabetes; (2) delay the onset of type I diabetes; and (3) allow for reduced rejection of transplanted islet cells from a non-diabetic subject to a diabetic subject; or (4) a combination of the above.

Thus, in an embodiment, a RAGE fusion protein of the present invention may be used to treat inflammation associated with transplantation of at least one of an organ, a tissue, or a plurality of cells from a first site to a second site. The first and second sites may be in different subjects, or in the same subject. In alternate embodiments, the transplanted cells, tissue or organ comprise cells of a pancreas, skin, liver, kidney, heart, lung, bone marrow, blood, bone, muscle, endothelial cells, artery, vein, cartilage, thyroid, nervous system, or stem cells. For example, administration of the RAGE fusion proteins of the present invention may be used to facilitate transplantation of islet cells from a first non-diabetic subject to a second diabetic subject.

In another embodiment, the present invention may provide a method of treating osteoporosis by administering to a subject a therapeutically effective amount of a RAGE fusion protein of the present invention (Zhou et al., J. Exp. Med., 203:1067-1080 (2006)). In an embodiment, the method of treating osteoporosis may further comprise the step of increasing bone density of the subject or reducing the rate of decrease in bone density of a subject.

Thus, in various selected embodiments, the present invention may provide a method for treatment of a disorder in a subject by administering to the subject a therapeutically effective amount of a fusion protein of the present invention. The subject treated using the fusion proteins of the present invention may be an animal. In an embodiment, the subject is a human.

A therapeutically effective amount may comprise an amount which is capable of preventing the interaction of a binding partner, ligand, modulator, or other agent with the non-immunoglobulin polypeptide as it normally functions in the subject. Accordingly, the amount will vary with the subject being treated. Administration of the compound may be hourly, daily, weekly, monthly, yearly, or as a single event. In various alternative embodiments, the effective amount of the fusion protein may range from about 1 ng/kg body weight to about 100 mg/kg body weight, or from about 10 μg/kg body weight to about 50 mg/kg body weight, or from about 100 μg/kg body weight to about 20 mg/kg body weight. The actual effective amount may be established by dose/response assays using methods standard in the art (Johnson et al., Diabetes. 42: 1179, (1993)). Thus, as is known to those in the art, the effective amount may depend on bioavailability, bioactivity, and biodegradability of the compound.

### Compositions

The present invention may comprise a composition comprising a fusion protein of the present invention mixed with a pharmaceutically acceptable carrier. Pharmaceutically acceptable carriers may comprise any of the standard pharmaceutically accepted carriers known in the art. In one embodiment, the pharmaceutical carrier may be a liquid and the fusion protein or nucleic acid construct may be in the form of a solution. In other embodiments, the pharmaceutically acceptable carrier may be a solid in the form of a powder, a lyophilized powder, or a tablet. Or, the pharmaceutical carrier may be a gel, suppository, or cream. In alternate embodiments, the carrier may comprise a liposome, a microcapsule, a polymer encapsulated cell, or a virus. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a trigyceride emulsion, various types of wetting agents, tablets, coated tablets and capsules.

Administration of the fusion proteins of the present invention may employ various routes. Administration of the fusion protein of the present invention may employ a parenteral route. For example, administration of the fusion protein of the present invention may employ a carrier suitable for intraperitoneal (IP) injection. In another embodiment, administration is intravenous (IV). The fusion protein may also be injected subcutaneously. Alternatively, the fusion protein may be administered orally, intranasally, or as an aerosol and thus, may comprise a carrier suitable for oral, intranasal or aerosol administration. In another embodiment, administration of the fusion protein is intra-arterial. In another embodiment, administration is sublingual. Also, administration may employ a time-release capsule. In these embodiments, the carrier may be suitable for IV, intraarterial, subcutaneous, sublingual, or time-release administration. For example, subcutaneous administration may be useful to treat chronic disorders when self-administration is desirable.

The pharmaceutical compositions may be in the form of a sterile injectable solution in a non-toxic parenterally acceptable solvent or vehicle. Among the acceptable vehicles and solvents that may be employed are water, Ringer's solution, 3-butanediol, isotonic sodium chloride solution, or aqueous buffers, as for example, physiologically acceptable citrate, acetate, glycine, histidine, phosphate, tris or succinate buffers. The injectable solution may contain stabilizers to protect against chemical degradation and aggregate formation. Stabilizers may include antioxidants such as butylated hydroxy anisole (BHA), and butylated hydroxy toluene (BHT), buffers (citrates, glycine, histidine) or surfactants (polysorbate 80, poloxamers). The solution may also contain antimicrobial preservatives, such as benzyl alcohol and parabens. The solution may also contain surfactants to reduce aggregation, such as Polysorbate 80, poloxomer, or other surfactants known in the art. The solution may also contain other additives, such as a sugar(s) or saline, to adjust the osmotic pressure of the composition to be similar to human blood.

The pharmaceutical compositions may be in the form of a sterile lyophilized powder for injection upon reconstitution with a diluent. The diluent can be water for injection, bacteriostatic water for injection, or sterile saline. The lyophilized powder may be produced by freeze drying a solution of the fusion protein to produce the protein in dry form. As is known in the art, the lyophilized protein generally has increased stability and a longer shelf life than a liquid solution of the protein. The lyophilized powder (cake) many contain a buffer to adjust the pH, as for example physiologically acceptable citrate, acetate, glycine, histidine, phosphate, tris or succinate buffer. The lyophilized powder may also contain lyoprotectants to maintain its physical and chemical stability. The commonly used lyoprotectants are non-reducing sugars and disaccharides such as sucrose, mannitol, or trehalose. The lyophilized powder may contain stabilizers to protect against chemical degradation and aggregate formation. Stabilizers may include, but are not limited to antioxidants (BHA, BHT), buffers (citrates, glycine, histidine), or surfactants (polysorbate 80, poloxamers). The lyophilized powder may also contain antimicrobial preservatives, such as benzyl alcohol and parabens. The lyophilized powder may also contain surfactants to reduce aggregation, such as, but not limited to, Polysorbate 80 and poloxomer. The lyophilized powder may also contain additives (e.g., sugars or saline) to adjust the osmotic pressure to be similar to human blood upon reconstitution of the powder. The lyophilized powder may also contain bulking agents, such as sugars and disaccharides.

The pharmaceutical compositions for injection may also be in the form of a oleaginous suspension. This suspension may be formulated according to the known methods using suitable dispersing or wetting agents and suspending agents described above. In addition, sterile, fixed oils are conveniently employed as solvent or suspending medium. For this purpose, any bland fixed oil may be employed using synthetic mono- or diglycerides. Also, oily suspensions may be formulated by suspending the active ingredient in a vegetable oil, for example arachis oil, olive oil, sesame oil or coconut oil, or in a mineral oil such as a liquid paraffin. For example, fatty acids such as oleic acid find use in the preparation of injectables. The oily suspensions may contain a thickening agent, for example beeswax, hard paraffin or cetyl alcohol. These compositions may be preserved by the addition of an anti-oxidant such as ascorbic acid.

The pharmaceutical compositions of the present invention may also be in the form of oil-in-water emulsions or aqueous suspensions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan.

Aqueous suspensions may also contain the active compounds in admixture with excipients. Such excipients may include suspending agents, for example sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Dispersible powders and granules suitable for preparation of an aqueous suspension by the addition of water may provide the active compound in admixture with a dispersing agent, suspending agent, and one or more preservatives. Suitable preservatives, dispersing agents, and suspending agents are described above.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions or suspensions containing the compounds of the invention may be used. Topical applications may also include mouth washes and gargles. Suitable preservatives, antioxidants such as BHA and BHT, dispersants, surfactants, or buffers may be used.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In certain embodiments, the compounds of the present invention may be modified to further retard clearance from the circulation by metabolic enzymes. In one embodiment, the compounds may be modified by the covalent attachment of water-soluble polymers such as polyethylene glycol (PEG), copolymers of PEG and polypropylene glycol, polyvinylpyrrolidone or polyproline, carboxymethyl cellulose, dextran, polyvinyl alcohol, and the like. Such modifications also may increase the compound's solubility in aqueous solution. Polymers such as PEG may be covalently attached to one or more reactive amino residues, sulfydryl residues or carboxyl residues. Numerous activated forms of PEG have been described, including active esters of carboxylic acid or carbonate derivatives, particularly those in which the leaving groups are N-hydroxsuccinimide, p-nitrophenol, imdazole or 1-hydroxy-2-nitrobenzene-3 sulfone for reaction with amino groups, multimode or halo acetyl derivatives for reaction with sulfhydryl groups, and amino hydrazine or hydrazide derivatives for reaction with carbohydrate groups. Additional methods for preparation of protein formulations which may be used with the fusion proteins of the present invention are described in U.S. Pat. Nos. 6,267,958, and 5,567,677.

In a further aspect of the present invention, the fusion proteins of the invention may be utilized in adjuvant therapeutic or combination therapeutic treatments with other known therapeutic agents. The following is a non-exhaustive listing of adjuvants and additional therapeutic agents which may be utilized in combination with the fusion protein modulators of the present invention:

Pharmacologic classifications of anticancer agents:
1. Alkylating agents: Cyclophosphamide, nitrosoureas, carboplatin, cisplatin, procarbazine
2. Antibiotics: Bleomycin, Daunorubicin, Doxorubicin
3. Antimetabolites: Methotrexate, Cytarabine, Fluorouracil, Azathioprine, 6-Mercaptopurine, and cytotoxic cancer chemotherapeutic agents
4. Plant alkaloids: Vinblastine, Vincristine, Etoposide, Paclitaxel,
5. Hormones: Tamoxifen, Octreotide acetate, Finasteride, Flutamide
6. Biologic response modifiers: Interferons, Interleukins

Pharmacologic classifications of treatment for Rheumatoid Arthritis
1. Analgesics: Aspirin
2. NSAIDs (Nonsteroidal anti-inflammatory drugs): Ibuprofen, Naproxen, Diclofenac
3. DMARDs (Disease-Modifying Antirheumatic drugs): Methotrexate, gold preparations, hydroxychloroquine, sulfasalazine
4. Biologic Response Modifiers, DMARDs: Etanercept, Infliximab Glucocorticoids, such as beclomethasone, methylprednisolone, betamethasone, prednisone, dexamethasone, and hydrocortisone

Pharmacologic classifications of treatment for Diabetes Mellitus
1. Sulfonylureas: Tolbutamide, Tolazamide, Glyburide, Glipizide
2. Biguanides: Metformin
3. Miscellaneous oral agents: Acarbose, Troglitazone
4. Insulin

Pharmacologic classifications of treatment for Alzheimer's Disease
1. Cholinesterase Inhibitor: Tacrine, Donepezil
2. Antipsychotics: Haloperidol, Thioridazine
3. Antidepressants: Desipramine, Fluoxetine, Trazodone, Paroxetine
4. Anticonvulsants: Carbamazepine, Valproic acid

In an embodiment, the compositions of the present invention may comprise a therapeutically effective amount of a fusion protein in combination with a single or multiple additional therapeutic agents. In addition to the agents heretofore described, the following therapeutic agents may be used in combination with the fusion proteins of the present invention: immunosuppressants, such as cyclosporin, tacrolimus, rapamycin and other FK-506 type immunosuppressants.

### EXAMPLES

Features and advantages of the inventive concept covered by the present invention are further illustrated in the examples which follow.

### Example 1A

### Production of RAGE Fusion Proteins

Two plasmids were constructed to express two RAGE-IgG fusion proteins. Both plasmids were constructed by ligating different lengths of a 5′ cDNA sequence from human RAGE with the same 3′ cDNA sequence from human IgG (γ1). These expression sequences (i.e., ligation products) were then inserted in pcDNA3.1 expression vector (Invitrogen, CA). The nucleic acid sequences that encode the RAGE fusion protein coding region are shown in FIGS. 5 and 6. For TTP-4000 RAGE fusion protein, the nucleic acid sequence from 1 to 753 (highlighted in bold) encodes the RAGE N-terminal protein sequence, whereas the nucleic acid sequence from 754 to 1386 encodes the IgG Fc protein sequence (FIG. 5). For TTP-3000, the nucleic acid sequence from 1 to 408 (highlighted in bold) encodes the RAGE N-terminal protein sequence, whereas the nucleic acid sequence from 409 to 1041 encodes the IgG Fc protein sequence (FIG. 6).

To produce the RAGE fusion proteins, the expression vectors comprising the nucleic acid sequences encoding the fusion proteins were transfected into CHO cells. Positive transformants were selected for neomycin resistance conferred by the plasmid and cloned. High producing clones as detected by Western Blot analysis of supernatant were expanded and the gene product was purified by affinity chromatography using Protein A columns. Expression was optimized so that cells were producing recombinant TTP-4000 at levels of about 1.3 grams per liter.

### Example 1B

### Removal of a Cryptic RNA Splice Site

A plasmid was constructed by ligating a 5′ cDNA sequence from human RAGE with a 3′ cDNA sequence from human IgG (γ1). PCR was used to amplify the cDNA. Further, on the 5′ end, the PCR primer added an Eco RI restriction enzyme site from cloning and a Kozak consensus translation initiation sequence. On the 3′ end, the PCR primer added a Xho I restriction just past the terminal codon. On the 3′ end, the PCR primer also included two silent base changes that remove a cryptic RNA splice site in the immunoglobulin portion near the terminal codon. The codon encoding for proline (residue 459 based on numbering in the protein sequence in SEQ ID NO: 16) was changed from CCG to CCC, and the codon encoding for glycine (residue 460 based on numbering in the protein sequence in SEQ ID NO: 16) was changed from GGT to GGG. The PCR fragment was digested with Eco RI and Xho I and then inserted into a retrovector plasmid (pCNS-newMCS-WPRE (new ori), available from Gala, Inc.) that had been digested with Mfe I (to form a compatible end with Eco RI) and digested with Xho I. The inserted portion of the cloned plasmid and cloning junctions were sequenced to ensure that no mutations occurred during cloning. To produce the RAGE-IgG fusion protein, the expression vector comprising the nucleic acid sequence SEQ ID NO: 21 was stably transfected in CHO cells.

The sequence of the isolated RAGE fusion protein TTP-4000 expressed by the transfected cells was confirmed by various characterization studies. It was found that the signal sequence encoded by the first 23 amino acids of SEQ ID NO: 16 was cleaved and the N-terminal residue was glutamine (Q) or pyroglutamic acid (pE) (SEQ ID NO: 18) or a mixture thereof. Characterization studies also showed glycosylation sites at N2 and N288 (based on numbering of the processed protein) and showed that the C_{H}3 region of the RAGE fusion protein may have its C-terminal residue cleaved off through a post-translational modification when expressed in this recombinant system.

### Example 2

### Method for Testing Activity of a RAGE-IgG1 Fusion Protein

A. In Vitro Ligand Binding:

Known RAGE ligands were coated onto the surface of Maxisorb plates at a concentration of 5 micrograms per well. Plates were incubated at 4° C. overnight. Following ligand incubation, plates were aspirated and a blocking buffer of 1% BSA in 50 mM imidizole buffer (pH 7.2) was added to the plates for 1 hour at room temperature. The plates were then aspirated and/or washed with wash buffer (20 mM Imidizole, 150 mM NaCl, 0.05% Tween-20, 5 mM CaCl₂ and 5 mM MgCl₂, pH 7.2). A solution of TTP-3000 (TT3) at an initial concentration of 1.082 mg/mL and a solution of TTP-4000 (TT4) at an initial concentration of 370 μg/mL were prepared. The RAGE fusion protein was added at increasing dilutions of the initial sample. The RAGE fusion protein was allowed to incubate with the immobilized ligand at 37° C. for one hour after which the plate was washed and assayed for binding of the RAGE fusion protein. Binding was detected by the addition of an immunodetection complex containing a monoclonal mouse anti-human IgG1 diluted 1:11,000 to a final assay concentration (FAC) of 21 ng/100 μL, a biotinylated goat anti-mouse IgG diluted 1:500, to a FAC of 500 ng/μL, and an avidin-linked alkaline phosphatase. The complex was incubated with the immobilized RAGE fusion protein for one hour at room temperature after which the plate was washed and the alkaline phosphatase substrate para-nitrophenylphosphate (PNPP) was added. Binding of the complex to the immobilized RAGE fusion protein was quantified by measuring conversion of PNPP to para-nitrophenol (PNP) which was measured spectrophotometrically at 405 nm.

As illustrated in FIG. 7, the RAGE fusion proteins TTP-4000 (TT4) and TTP-3000 (TT3) specifically interact with known RAGE ligands amyloid-beta (Abeta), S100b (S100), and amphoterin (Ampho). In the absence of ligand, i.e., BSA coating alone (BSA or BSA+wash) there was no increase in absorbance over levels attributable to non-specific binding of the immunodetection complex. Where amyloid beta is used as the labeled ligand it may be necessary to preincubate the amyloid beta before the assay. Preincubation may allow the amyloid beta to self-aggregate into pleated sheet form, as amyloid beta may preferentially bind to RAGE in the form of a pleated sheet.

Additional evidence for a specific interaction between RAGE fusion proteins TTP-4000 and TTP-3000 with RAGE ligands is exemplified in studies showing that a RAGE ligand is able to effectively compete with a known RAGE ligand for binding to the RAGE fusion proteins. In these studies, amyloid-beta (A-beta) was immobilized on a Maxisorb plate and RAGE fusion protein added as described above. In addition, a RAGE ligand was added to some of the wells at the same time as the RAGE fusion protein.

It was found that the RAGE ligand could block binding of TTP-4000 (TT4) by about 25% to 30% where TTP-4000 was present at 123 μg/mL (1:3 dilution, FIG. 8). When the initial solution of TTP-4000 was diluted by a factor of 10 or 30 (1:10 or 1:30), binding of the RAGE fusion protein to the immobilized ligand was completely inhibited by the RAGE ligand. Similarly, the RAGE ligand blocked binding of TTP-3000 (TT3) by about 50% where TTP-3000 was present at 360 μg/mL (1:3 dilution, FIG. 9). When the initial solution of TTP-3000 was diluted by a factor of 10 (1:10), binding of the RAGE fusion protein to the immobilized ligand was completely inhibited by the RAGE ligand. Thus, specificity of binding of the RAGE fusion protein to the RAGE ligand was dose dependent. Also, as shown in FIGS. 8 and 9, there was essentially no binding detected in the absence of RAGE fusion protein, i.e., using only the immunodetection complex (“Complex alone”).

B. Effect of RAGE Fusion Proteins in a Cell Based Assays

Previous work has shown that the myeloid THP-1 cells may secrete TNF-α in response to RAGE ligands. In this assay, THP-1 cells were cultured in RPMI-1640 media supplemented with 10% FBS using a protocol provided by ATCC. The cells were induced to secrete TNF-α via stimulation of RAGE with 0.1 mg/ml S100b both in the absence and the presence of the RAGE fusion proteins TTP-3000 (TT3) or TTP-4000 (TT4) (10 μg), sRAGE (10 μg), and a human IgG (10 μg) (i.e., as a negative control). The amount of TNF-α secreted by the THP-1 cells was measured 24 hours after the addition of the proteins to the cell culture using a commercially available ELISA kit for TNF-α (R&D Systems, Minneapolis, Minn.). The results in FIG. 10 demonstrate that the RAGE fusion proteins inhibit the S100b/RAGE-induced production of TNF-α in these cells. As shown in FIG. 10, upon addition of 10 μg TTP-3000 or TTP-4000 RAGE fusion protein, induction of TNF-α by S100b (0.1 mg/ml FAC) was reduced by about 45% to 70%, respectively. Fusion protein TTP-4000 may be at least as effective in blocking S100b induction of TNF-α as is sRAGE (FIG. 10). Specificity of the inhibition of TNF-α induction by TTP-4000 and TTP-3000 for RAGE sequences of the RAGE fusion protein is shown by an experiment in which IgG alone was added to S100b stimulated cells. It can be seen that the addition of IgG, i.e., human IgG without the RAGE sequence (Sigma human IgG added at 10 μg/well), and S100b to the assay shows the same levels of TNF-α as S100b alone.

### Example 3

### Pharmacokinetic Profile of TTP-4000

To determine whether TTP-4000 would have a superior pharmacokinetic profile as compared to human sRAGE, rats and nonhuman primates were given an intravenous (IV) injection of TTP-4000 (5 mg/kg) and then plasma was assessed for the presence of TTP-4000. In these experiments, two naïve male monkeys received a single IV bolus dose of TTP-4000 (5 mg/ml/kg) in a peripheral vein followed by an approximate 1.0 milliliter (mL) saline flush. Blood samples (approximately 1.0 mL) were collected at pre-dose (i.e., prior to injection of the TTP-4000), or at 0.083, 0.25, 0.5, 2, 4, 8, 12, 24, 48, 72, 96, 120, 168, 240, 288, and 336 hours post dose into tubes containing (lithium heparin). Following collection, the tubes were placed on wet ice (maximum 30 minutes) until centrifugation under refrigeration (at 2 to 8° C.) at 1500×g for 15 minutes. Each harvested plasma sample was then stored frozen (−70° C.±10° C.) until assayed for RAGE polypeptide using an ELISA at various time-points following the injection, as described in Example 6.

The kinetic profile shown in FIG. 11 reveals that once TTP-4000 has saturated its ligands as evidenced by the fairly steep slope of the alpha phase in 2 animals, it retains a terminal half-life of greater than 300 hours. This half-life is significantly greater than the half-life of human sRAGE in plasma (generally about 2 hours) and provides an opportunity for single injections for acute and semi-chronic indications. In FIG. 11 each curve represents a different animal under the same experimental conditions.

### Example 4

### TTP-4000 Fc Activation

Experiments were performed to measure the activation of the Fc receptor by RAGE fusion protein TTP-4000 as compared to human IgG. Fc receptor activation was measured by measuring TNF-α secretion from THP-1 cells that express the Fc receptor. In these experiments, a 96 well plate was coated with 10 μg/well TTP-4000 or human IgG. Fc stimulation results in TNF-α secretion. The amount of TNF-α was measured by an Enzyme Linked Immunoabsorbent Assay (ELISA).

Thus, in this assay, the myeloid cell line, THP-1 (ATTC #TIB-202) was maintained in RPMI-1640 media supplemented with 10% fetal bovine serum per ATCC instructions. Typically, 40,000-80,000 cells per well were induced to secrete TNF-alpha via Fc receptor stimulation by precoating the well with 10 ug/well of either heat aggregated (63° C. for 30 min) TTP-4000 or human IgG1. The amount of TNF-alpha secreted by the THP-1 cells was measured in supernatants collected from 24 hours cultures of cells in the treated wells using a commercially available TNF ELISA kit (R&D Systems, Minneapolis, Minn. #DTA00C) per instructions.

Results are shown in FIG. 12 where it can be seen that TTP-4000 generates less than 2 ng/well TNF and IgG generated greater than 40 ng/well.

### Example 5

### In Vivo Activity of TTP-4000

The activity of TTP-4000 was compared to sRAGE in several in vivo models of human disease.

A. TTP-4000 in an Animal Model of Restenosis

The RAGE fusion protein TTP-4000 was evaluated in a diabetic rat model of restenosis which involved measuring smooth muscle proliferation and intimal expansion 21 days following vascular injury. In these experiments, balloon injury of left common carotid artery was performed in Zucker diabetic and nondiabetic rats using standard procedure. A loading dose (3 mg/rat) of IgG, TTP-4000 or phosphate buffered saline (PBS) was administered intraperitoneally (IP) one day prior injury. A maintenance dose was delivered every other day until day 7 after injury (i.e., at day 1, 3, 5 and 7 after injury). The maintenance dose was high=1 mg/animal for one group, or low=0.3 mg/animal for the second group. To measure vascular smooth muscle cell (VSMC) proliferation, animals were sacrificed at 4 days and 21 days after injury.

For the measurement of cell proliferation, 4 day animals received intraperitoneal injection of bromodeoxyuridine (BrDdU) 50 mg/kg at 18, 12, and 2 hours before euthanasia. After sacrifice, the entire left and right carotid arteries were harvested. Specimens were stored in Histochoice for at least 24 hours before embedding. Assessment of VSMC proliferation was performed using mouse anti-BrdU monoclonal antibody. A fluorescence labeled goat anti-mouse secondary antibody was applied. The number of BrdU-positive nuclei per section were counted by two observers blinded to the treatment regimens.

The remaining rats were sacrificed at 21 days for morphometric analysis. Morphometric analyses were performed by an observer blinded to the study groups, using computerized digital microscopic planimetry software Image-Pro Plus on serial sections, (5 mm apart) carotid arteries stained by Van Gieson staining All data were expressed as mean±SD. Statistical analysis was performed with use of SPSS software. Continuous variables were compared using unpaired t tests. A values of P≦0.05 was considered to be statistically significant. As seen in Table 1, TTP-4000 treatment significantly reduced the intima/media ratio and vascular smooth muscle cell proliferation in a dose-responsive fashion.

B. Efficacy of TTP-4000 in an Animal Model of Stroke

TTP-4000 was also compared to sRAGE in a disease relevant animal model of stroke. In this model, the middle carotid artery of a mouse was ligated for 1 hour followed by 23 hours of reperfusion at which point the mice were sacrificed and the area of the infarct in the brain was assessed. Mice were treated with sRAGE or TTP-4000 or control immunoglobulin just prior to reperfusion.

In these experiments, male C57BL/6 were injected with vehicle at 250 μl/mouse or TTP test articles (TTP-3000, TTP-4000 at 250 μl/mouse). Mice were injected intraperitoneally, 1 hour after the initiation of ischemia. Mice were subjected to one hour of cerebral ischemia followed by 24 hours of reperfusion. To induce ischemia, each mouse was anesthetized and body temperature was maintained at 36-37° C. by external warming. The left common carotid artery (CCA) was exposed through a midline incision in the neck. A microsurgical clip was placed around the origin of the internal carotid artery (ICA). The distal end of the ECA was ligated with silk and transected. A 6-0 silk was tied loosely around the ECA stump. The fire-polished tip of a nylon suture was gently inserted into the ECA stump. The loop of the 6-0 silk was tightened around the stump and the nylon suture was advanced into and through the internal carotid artery (ICA), until it rested in the anterior cerebral artery, thereby occluding the anterior communicating and middle cerebral arteries. After the nylon suture had been in place for 1 hour, the animal was re-anesthetized, rectal temperature was recorded and the suture was removed and the incision closed.

Infarct volume was determined by anesthetizing the animals with an intraperitoneal injection of sodium pentobarbital (50 mg/kg) and then removing the brains. The brains were then sectioned into four 2-mm sections through the infracted region and placed in 2% triphenyltetrazolium chloride (TTC) for 30 minutes. After, the sections were placed in 4% paraformaldehyde over night. The infarct area in each section was determined with a computer-assisted image analysis system, consisting of a Power Macintosh computer equipped with a Quick Capture frame grabber card, Hitachi CCD camera mounted on a camera stand. NIH Image Analysis Software, v. 1.55 was used. The images were captured and the total area of infarct was determined over the sections. A single operator blinded to treatment status performed all measurements. Summing the infarct volumes of the sections calculated the total infarct volume. The results are expressed as the mean±standard deviation (SD). The significance of difference in the infarct volume data was analyzed using a t-test. As illustrated by the data in Table 2, TTP-4000 was more efficacious than sRAGE in limiting the area of infarct in these animals suggesting that TTP-4000, because of its better half-life in plasma, was able to maintain greater protection in these mice.

### Example 6

### Detection of RAGE Fusion Protein by ELISA

Initially, 50 uL of the RAGE specific monoclonal antibody 1HB1011 at a concentration of 10 ug/mL in 1×PBS pH 7.3 is coated on plates via overnight incubation. When ready for use, plates are washed three times with 300 uL of 1× Imidazole-Tween wash buffer and blocked with 1% BSA. The samples (diluted) and standard dilutions of known TTP-4000 dilutions are added at 100 uL final volume. The samples are allowed to incubate at room temperature for one hour. After incubation, the plates are plates are washed three times. A Goat Anti-human IgG1 1 (Sigma A3312) AP conjugate in 1×PBS with 1% BSA is added and allowed to incubate at room temperature for 1 hour. The plates are washed three times. Color was elucidated with paranitrophenylphosphate.

### Example 7

### Quantification of RAGE Ligand Binding to RAGE Fusion Protein

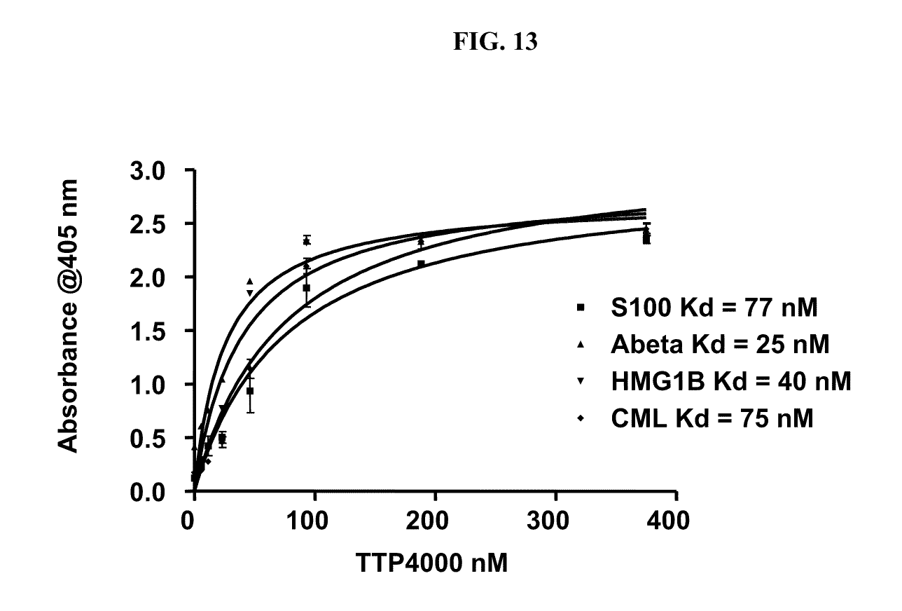

Saturation-binding of TTP-4000 to various immobilized known RAGE ligands was performed (FIG. 13) using the ELISA portion of the protocol of Example 6. The ligands were immobilized on a microtiter plate and incubated in the presence of increasing concentrations of RAGE fusion protein from 0 to 360 nM. The RAGE fusion protein-ligand interaction is detected using a polyclonal antibody conjugated with alkaline phosphatase that is specific for the IgG portion of the fusion chimera. Relative Kds were calculated using Graphpad Prizm software and match with established literature values of RAGE-RAGE ligand values. HMG1B=Ampoterin, CML=Carboxymethyl Lysine, A beta=Amyloid beta 1-40.

The foregoing is considered as illustrative only of the principal of the invention. Since numerous modifications and changes will readily occur to those skilled in the art, it is not intended to limit the invention to the exact embodiments shown and described, and all suitable modifications and equivalents falling within the scope of the appended claims are deemed within the present inventive concept.

What is claimed is:

1. A fusion protein comprising:
(a) an immunoglobulin polypeptide from which the hinge region has been removed; and
(b) a non-immunoglobulin polypeptide comprising an uninterrupted portion of a CTLA-4 protein, wherein the uninterrupted portion of the CTLA-4 protein comprises a first non-immunoglobulin polypeptide domain directly linked to the N-terminal end of a first interdomain linker,
wherein the immunoglobulin polypeptide is operably linked to the C-terminal end of the first interdomain linker, and
wherein the first non-immunoglobulin polypeptide domain and the first interdomain linker comprise an uninterrupted portion of a polypeptide derived from CTLA-4.

2. The fusion protein of claim 1, wherein the immunoglobulin polypeptide is directly linked to the C-terminal end of the first interdomain linker.

3. The fusion protein of claim 1, wherein the immunoglobulin polypeptide comprises a portion of the C_{H}2 domain and the C_{H}3 domain of an immunoglobulin.

4. The fusion protein of claim 1, comprising a human immunoglobulin polypeptide and a human CTLA-4 polypeptide.

5. The fusion protein of claim 1, wherein the non-immunoglobulin polypeptide comprises a ligand binding domain or a ligand binding site.

6. The fusion protein of claim 1, wherein the non-immunoglobulin polypeptide further comprises a second non-immunoglobulin polypeptide domain that is operably linked to the N-terminal end of the first non-immunoglobulin polypeptide domain.

7. The fusion protein of claim 1, wherein the first interdomain linker does not promote binding of the fusion protein to a Fc receptor (FcR) at a therapeutically effective concentration of the fusion protein.

8. The fusion protein of claim 1, wherein the C-terminal amino acid of the first interdomain linker is directly linked to the N-terminal amino acid of a polypeptide comprising a C_{H}2 domain from which the hinge region has been removed.

9. The fusion protein of claim 1, wherein the immunoglobulin polypeptide is an Fc fragment from which the Fc hinge region has been removed and the first interdomain linker is positioned in place of the immunoglobulin Fc hinge region.

10. A composition comprising the fusion protein of claim 1 and a pharmaceutically acceptable carrier.

11. The composition of claim 10, wherein the pharmaceutically acceptable carrier is suitable for at least one of intravenous, intraperitoneal, or subcutaneous administration.

12. The composition of claim 10, wherein the fusion protein is formulated as a sterile lyophilized powder.

13. The composition of claim 10, wherein the immunoglobulin polypeptide is directly linked to the C-terminal end of the first interdomain linker.


SEQUENCE LISTING (28 sequences)

SEQ ID NO: 1 - protein, 404 aa, Homo sapiens
  human receptor for advanced glycation endproducts (RAGE)
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - DNA, 753 bp, Homo sapiens
  DNA encoding human receptor for advanced glycation endproducts (RAGE) amino acids 1-251
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcct

SEQ ID NO: 3 - protein, 7 aa, Artificial
  synthetic receptor for advanced glycation endproducts (RAGE) interdomain linker
VYQIPGK

SEQ ID NO: 4 - protein, 30 aa, Artificial
  synthetic receptor for advanced glycation endproducts (RAGE) second interdomain linker
TAPIQPRVWEPVPLEEVQLVVEPEGGAVAP

SEQ ID NO: 5 - protein, 20 aa, Homo sapiens
  synthetic receptor for advanced glycation endproducts (RAGE) third interdomain linker
VYQIPGKPEIVDSASELTAG

SEQ ID NO: 6 - protein, 5 aa, Artificial
  synthetic receptor for advanced glycation endproducts (RAGE) fourth interdomain linker
TAPIQ

SEQ ID NO: 7 - protein, 25 aa, Artificial
  synthetic receptor for advanced glycation endproducts (RAGE) fifth interdomain linker
ISIIEPGEEGPTAGSVGGSGLGTLA

SEQ ID NO: 8 - protein, 210 aa, Homo sapiens
  human IgG1 C-H2 and C-H3 domains without hinge region
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 633 bp, Homo sapiens
  human IgG1 C-H2 and C-H3 domains without hinge region
ccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - DNA, 633 bp, Artificial
  synthetic human IgG1 C-H2 and C-H3 domains without hinge region with modified C-terminus to remove cryptic RNA splice site
ccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 11 - protein, 220 aa, Homo sapiens
  human IgG C-H2 and C-H3 domains including portion of middle and lower hinge regions
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 663 bp, Homo sapiens
  human IgG C-H2 and C-H3 domains including portion of middle and lower hinge regions
ccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - DNA, 663 bp, Artificial
  synthetic IgG C-H2 and C-H3 domains including portion of middle and lower hinge regions modified to remove cryptic RNA splice site
ccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 14 - protein, 103 aa, Homo sapiens
  human IgG C-H2 domain without lower and middle hinge regions
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 107 aa, Homo sapiens
  human IgG C-H3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 461 aa, Artificial
  synthetic four domain RAGE-Fc IgG fusion protein
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 346 aa, Artificial
  synthetic three domain RAGE-Fc IgG fusion protein
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 438 aa, Artificial
  synthetic four domain RAGE-Fc IgG fusion protein having signal sequence removed
ENITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 323 aa, Artificial
  synthetic three domain RAGE-Fc IgG fusion protein having signal sequence removed
ENITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1386 bp, Artificial
  synthetic four domain RAGE-Fc IgG1 fusion protein (TTP-4000)
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - DNA, 1386 bp, Artificial
  synthetic four domain RAGE-Fc IgG1 fusion protein (TTP-4000) modified to remove cryptic RNA splice site
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 22 - DNA, 1041 bp, Artificial
  synthetic three domain RAGE-Fc IgG1 fusion protein (TTP-3000)
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 23 - DNA, 1041 bp, Artificial
  synthetic four domain RAGE-Fc IgG1 fusion protein (TTP-4000) modified to remove cryptic RNA splice site
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 24 - protein, 22 aa, Artificial
  synthetic human IgG1 hinge region
EPKSCDKTHTCPPCPAPELLGG

SEQ ID NO: 25 - protein, 10 aa, Artificial
  synthetic human IgG1 hinge region upper hinge
EPKSCDKTHT

SEQ ID NO: 26 - protein, 5 aa, Artificial
  synthetic human IgG1 hinge region middle hinge
CPPCP

SEQ ID NO: 27 - protein, 7 aa, Artificial
  synthetic human IgG1 hinge region lower hinge
APELLGG

SEQ ID NO: 28 - protein, 4 aa, Artificial
  synthetic human IgG1 motif of lower hinge region of Fc domain Fc-FcR binding sequence, Fc receptor domain
LLGG